(19) United States
(12) Patent Application Publication
(10) Pub. No.: US 2025/0236853 A1
(43) Pub. Date: Jul. 24, 2025

(54) Reverse Transcriptases and Related Methods

(71) Applicant: New England Biolabs, Inc., Ipswich, MA (US)

(72) Inventors: Jackson A. Buss, Groveland, MA (US); Haruichi Asahara, Ipswich, MA (US); Guoping Ren, Danvers, MA (US); Yan Xu, Hamilton, MA (US); Sean R. Johnson, Beverly, MA (US); Jennifer Ong, Salem, MA (US); Gregory Patton, Peabody, MA (US); Vladimir Potapov, Auburndale, MA (US); Evan Janzen, Danvers, MA (US)

(73) Assignee: New England Biolabs, Inc., Ipswich, MA (US)

(21) Appl. No.: 19/028,646

(22) Filed: Jan. 17, 2025

Related U.S. Application Data
(60) Provisional application No. 63/622,710, filed on Jan. 19, 2024.

(51) Int. Cl. C12N 9/12; C12N 15/10; C12Q 1/6851

(52) U.S. Cl. CPC C12N 9/1276; C12N 15/1096; C12Q 1/6851; C07K 2319/20; C12Y 207/07049

(57) ABSTRACT

Reverse transcriptases that may have an improvement in one or more properties are provided. For example, the present reverse transcriptases are believed to be more efficient than other reverse transcriptases, particularly in the presence of substances that typically inhibit reverse transcriptases. Also provided are related compositions, kits, and methods for using the reverse transcriptases.

### CROSS-REFERENCING

This application claims the benefit of provisional application Ser. No. 63/622,710, filed on Jan. 19, 2024, which application is incorporated herein in its entirety.

### SEQUENCE LISTING

This application is filed with a Sequence Listing in electronic form as a Sequence Listing XML, “NEB-478-US” created on Jan. 17, 2025, and having a size of 10,585 bytes. The contents of the Sequence Listing XML are incorporated by reference herein in their entirety.

### BACKGROUND

Reverse transcriptases (RTs) are multi-functional enzymes that typically have multiple enzymatic activities, including an RNA-dependent DNA polymerization activity, a DNA-dependent DNA polymerization activity, and an RNaseH activity that catalyzes the cleavage of RNA in RNA-DNA hybrids. These enzymes, which are used to synthesize complementary DNA (cDNA) using RNA as a template, were first identified in RNA viruses. Subsequently, reverse transcriptases have been isolated and purified directly from virus particles, cells, and tissues (e.g., see Kacian et al., 1971, Biochim. Biophys. Acta 46:365-83; Yang et al., 1972, Biochem. Biophys. Res. Comm. 47:505-11; Gerard et al., 1975, J. Virol. 15:785-97; Liu et al., 1977, Arch. Virol. 55 187-200; Kato et al., 1984, J. Virol. Methods 9:325-39; Luke et al., 1990, Biochem. 29:1764-69 and Le Grice et al., 1991, J. Virol. 65:7004-07). A variety of RTs are commercially available and are routinely used in research and diagnostic applications such as PCR tests and RNA sequencing. These include naturally occurring RTs such as Moloney murine leukemia virus (MMLV) RT and derivatives engineered to have improvements, such as greater thermostability. However, there remains a need for improved RTs that work under challenging conditions. For example, the presence of certain sample constituents, such as salts and substances present in medical and environmental samples, can reduce the efficiency of RTs. Reduced RT efficiency leads to a reduced amount of copied DNA generated and even to false positive results in quantitative PCR tests. Thus, it would be desirable to obtain new RTs that can synthesize DNA under a variety of conditions, such as in the presence of substances that inhibit activity of some RTs.

### SUMMARY

Provided herein are reverse transcriptases comprising an amino acid sequence that is at least 90% identical to the amino acid sequence of SEQ ID NO:1 or SEQ ID NO:2. In some embodiments, the reverse transcriptase has an improvement in one or more properties as compared to other known reverse transcriptases. For example, the present reverse transcriptase may be more efficient than other reverse transcriptases, particularly in the presence of substances that typically inhibit reverse transcriptases. Also provided are variants of the reverse transcriptases and fusion proteins of the reverse transcriptases, which have reverse transcriptase activity. Kits, reaction mixes and methods that include the reverse transcriptase are also provided.

These and other features of the present teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows first strand cDNA synthesis activity of embodiments of engineered reverse transcriptases described herein (ERT1 (SEQ ID NO:1), ERT2 (SEQ ID NO:2), ERT3 (SEQ ID NO: 3) and ERT4 (SEQ ID NO:4)) relative to a control RT (CAV RT1) under stringent, high salt conditions.

FIG. 2 shows salt tolerance of an embodiment of reverse transcriptase described herein (ERT1) in high yield cDNA synthesis, relative to a control RT (CAV RT1), from RNA templates of 1 kb or 4 kb in length.

FIGS. 3A-3D show inhibitor tolerance of an embodiment of reverse transcriptase described herein (ERT1); FIG. 3A shows cDNA synthesis relative to a control RT in the presence of various salt conditions; FIG. 3B shows cDNA synthesis relative to a control RT in the presence of various chemicals; FIG. 3C shows cDNA synthesis relative to a control RT in the presence of various dyes and lysis reagents; FIG. 3D shows cDNA synthesis relative to a control RT in the presence of various environmental and animal sample components.

FIG. 4 shows high yield cDNA synthesis for long cDNA under various reaction conditions of an embodiment of reverse transcriptase described herein (ERT1).

FIGS. 5A-5D shows results from a one-step RT-qPCR assay using an embodiment of reverse transcriptase described herein (ERT1) relative to a control RT (CAV RT1). FIG. 5A shows amplification of actin; FIG. 5B shows amplification of SMG; FIG. 5C shows amplification of TUB; FIG. 5D shows plots of the results indicating efficiency of ERT1 relative to a control RT.

FIG. 6 shows results from a two-step RT-qPCR assay using an embodiment of reverse transcriptase described herein (ERT1) relative to a control RT (CAV RT1), in which a variety of target nucleic acids were amplified.

FIGS. 7A-7C shows results from a two-step RT-qPCR assay using an embodiment of reverse transcriptase described herein (ERT1) (FIG. 7B) relative to a control RT (CAV RT1) (FIG. 7A); FIG. 7C shows efficiency of cDNA synthesis.

FIG. 8 shows results from a 5-plex two-step RT-qPCR assay using an embodiment of reverse transcriptase described herein (ERT1) relative to a control RT (CAV RT1).

FIG. 9 shows results from a two-step RT-qPCR assay using an embodiment of reverse transcriptase described herein (ERT1) relative to a control RT (CAV RT1), indicating even coverage of the target nucleic acid.

FIG. 10 shows template switching activity of an embodiment of reverse transcriptase described herein (ERT1).

FIG. 11 shows a plot of full-length cDNA product formation as a function of temperature for an embodiment of a reverse transcriptase described herein (ERT1) (open square) and commercially available RT (closed square).

FIG. 12A shows amplified cDNA produced from total RNA through the template switching activity of an embodiment of a reverse transcriptase described herein (ERT1) in the absence (−) or presence (+) of T4 Gene 32 Protein (GP32). FIG. 12B shows the corresponding cDNA yield for each sample.

FIG. 13 is a graph of average normalized transcript coverage from sequencing cDNA produced by ERT1 or the template switching (TS) control enzyme in template switching reactions of cell-derived total RNA. Shown is the normalized sequencing read depth for each position (percentile) of the 1000 most-abundant transcripts. A normalized coverage of 1 across all percentiles would represent even coverage with no bias for specific regions of transcripts. Reverse transcription in these reactions is initiated at the 3′ end of transcripts (100 percent on the graph) by using primers that anneal to poly(A) tails of polyadenylated RNA sequences.

### DESCRIPTION

Provided herein are engineered reverse transcriptase enzymes (RTs) that can have improvements in one or more properties as compared to one or more known RTs. For example, the present RTs are believed to be more efficient in the presence of sample constituents that can inhibit reverse transcriptase activity (e.g., salts) relative to commercially available RTs. It is also believed that the described RTs can produce copied DNA in two-step RT-qPCR within short reaction times relative to commercially available RTs.

Inhibitory compounds can exist in RNA and/or DNA samples even after purification, causing reduced efficiency of cDNA synthesis and possibly resulting in false RT-PCR and RT-qPCR results. As described in Example 3, an embodiment of the engineered reverse transcriptases described herein has surprisingly robust polynucleotide extension activity in the presence of a variety of compounds that inhibit known reverse transcriptases, such as salts, chemicals, sample-processing reagents, dyes, and compounds naturally present in environmental and animal samples.

Although embodiments of the disclosure are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the embodiments, specific terminology will be resorted to for the sake of clarity.

It must also be noted that, as used in the specification and the appended claims, the singular forms “a,” “an” and “the” include plural referents unless the context clearly dictates otherwise. For example, reference to a sheet or portion is intended also to include the manufacturing of a plurality of sheets or portions. References to a sheet containing “a” constituent is intended to include other constituents in addition to the one named.

All publications cited herein are incorporated by reference herein.

Also, in describing the embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another embodiment includes from the one particular value and/or to the other particular value. “Comprising” or “containing” or “including” mean that at least the named compound, element, particle, or method step is present in the composition or article or method, but does not exclude the presence of other compounds, materials, particles, method steps, even if the other such compounds, material, particles, method steps have the same function as what is named.

The term “non-naturally occurring” used in reference to a polypeptide or composition described herein means that the polypeptide or composition does not exist in nature. A “non-naturally occurring” composition can differ from naturally occurring compositions in one or more of the following respects: (a) having components that are not combined in nature; (b) having components in concentrations not found in nature; (c) omitting one or more components otherwise found in naturally occurring compositions; (d) having a form not found in nature, e.g., dried, freeze dried, crystalline, aqueous; and (e) having one or more additional components beyond those found in nature (e.g., buffering agents, a detergent, a dye, a solvent or a preservative). The RT compositions, reaction mixtures, and mixtures formed when performing the described methods are examples of non-naturally occurring compositions; the RTs described herein are examples of non-naturally occurring polypeptides.

The term “position,” when used in reference to an amino acid, means the place such an amino acid occupies in the primary sequence of a polypeptide numbered from its amino terminus to its carboxy terminus. A position in one primary sequence can correspond to a position in a second primary sequence, for example, where the two positions are opposite one another when the two primary sequences are aligned using an alignment algorithm (e.g., BLAST (Journal of Molecular Biology. 215 (3): 403-410) using default parameters (e.g., expect threshold 0.05, word size 3, max matches in a query range 0, matrix BLOSUM62, Gap existence 11 extension 1, and conditional compositional score matrix adjustment) or custom parameters). An amino acid position in one sequence can correspond to a position within a functionally equivalent motif or structural motif that can be identified within one or more other sequence(s) in a database by alignment of the motifs. Analogously, with reference to a nucleotide, “position” means the place such nucleotide occupies in the nucleotide sequence of an oligonucleotide or polynucleotide numbered from its 5′ end to its 3′ end.

This disclosure relates to RTs designed using computational approaches. These engineered RTs differ from the well-studied MMLV RT in that they have C- and N-terminal truncations, amino acid insertions, and/or at least about 20% of amino acids of the RTs differ from corresponding positions in the MMLV reverse transcriptase. The MMLV RT has been crystallized (see, e.g., Das et al., Structure. 2004 12:819-29), the structure-functional relationships in MMLV RT have been studied (see, e.g., Cote et al., Virus Res. 2008 134:186-202, Georgiadis et al., Structure. 1995 3:879-92 and Crowther et al., Proteins 2004 57:15-26) and many mutations in MMLV RT are known (see, e.g., Yasukawa et al., J. Biotechnol. 2010 150:299-306, Arezo et al Nucleic Acids Res. 2009 37:473-81 and Konishi et al., Biochem. Biophys. Res. Commun. 2014 454:269-74, among many others).

As used herein, the term “reverse transcriptase” means a DNA polymerase that can copy first-strand cDNA from an RNA template, and in some cases, a DNA template, via its polymerase domain. Such enzymes are commonly referred to as RNA-directed DNA polymerases and have IUBMB activity EC 2.7.7.49. In some cases, a reverse transcriptase can copy a complementary DNA strand using either single-stranded RNA or DNA as a template. The RTs described herein have reverse transcriptase activity. The RTs described herein can use RNA and/or DNA as a template. Thus, the RTs described herein can copy first-strand DNA from RNA template and can copy second-strand DNA from the resulting cDNA, e.g., for applications such as library preparation and RT-PCR.

The RTs described herein may have template switching activity. The term “template switching” means a reverse transcription reaction in which the reverse transcriptase switches template from an RNA molecule to a synthetic oligonucleotide (which usually contains two or three Gs at its 3′ end, thereby copying the sequence of the synthetic oligonucleotide onto the end of the cDNA). Template switching is described, for example, in Matz et al., Nucl. Acids Res. 1999 27:1558-1560 and Wu et al., Nat Methods. 2014 11:41-6. In template switching, a primer hybridizes to an RNA template. This primer serves as a primer for an RT that copies the RNA template to form a complementary DNA product. In copying the RNA template, the RT commonly travels beyond the 5′ end of the RNA template to add non-template nucleotides to the 3′ end of the DNA product (typically Cs). Upon addition of an oligonucleotide that has ribonucleotides or deoxyribonucleotides that are complementary to the non-template nucleotides added onto the DNA product (e.g., a “template switching” oligonucleotide that typically has two or three G's at its 3′ end), the RT will jump templates from the RNA template to the oligonucleotide template, thereby producing a DNA product that has the complement of the template switching oligonucleotide at its 3′ end. Example 10 describes exemplary template switching activity of an RT disclosed herein. The template switching activity of an RT described herein is useful in applications where it is desirable to add a sequence to a DNA product, such as for mRNA sequencing, targeted RNA sequencing, rare transcript detection, single-cell RNA sequencing, cDNA library construction, diagnostic applications.

The RTs described herein may have RNase H activity or may lack RNAse H activity. The term “RNAse H activity” means an enzymatic activity that hydrolyzes RNA in RNA/DNA hybrid. Many reverse transcriptases have an RNAse H activity that can be inactivated by truncation or by substitution. The RTs described herein can lack RNAse H activity. For example, the RT of SEQ ID NO: 1 contains amino acids at locations expected to reduce or abolish RNAse H activity (e.g., G at position 502; Q at position 540, and N at position 561) and likewise the RT of SEQ ID NO:2 has G at position 500, Q at position 538 and N at position 559. These amino acids can be mutated to increase RNAse H activity. For example, the following substitutions could restore RNAse H activity: for SEQ ID NO:1, D at position 502, E at position 540, and D at position 561 and likewise for SEQ ID NO:2, D at position 500, E at position 538 and D at position 559.

Provided herein a reverse transcriptase (RT) comprising (a) an amino acid sequence having at least 90% identity with SEQ ID NO:1, or (b) an amino acid sequence having at least 90% identity with SEQ ID NO:2.

There is also provided a reverse transcriptase (RT) consisting of an amino acid sequence containing an N-terminal start sequence (e.g., a methionine) and (a) an amino acid sequence having at least 90% identity with SEQ ID NO:1, or (b) an amino acid sequence having at least 90% identity with SEQ ID NO:2. It is understood that a polypeptide consisting of a particular sequence can further contain one or more amino acids required for translation of the amino acid sequence into a polypeptide. Thus, in some embodiments, in which the RT consists of a defined amino acid sequence, it is understood that the RT may further contain a start codon, e.g., methionine, to permit its expression.

In some embodiments, the RT comprises an amino acid sequence that has at least 90% sequence identity to SEQ ID NO: 1 (e.g., at least 93%, at least 95%, at least 96%, at least 97%, at least 98%, or at least 99% sequence identity to SEQ ID NO: 1) but less than 100% sequence identity to SEQ ID NO: 1. In an embodiment the RT comprises an amino acid sequence that has at least 95% sequence identity to SEQ ID NO:1. In an embodiment, the RT comprises an amino acid sequence that is identical to SEQ ID NO:1.

In some embodiments, the RT comprises an amino acid sequence that has at least 90% sequence identity (e.g., at least 93%, at least 95%, at least 96%, at least 97%, at least 98% or at least 99% sequence identity) to SEQ ID NO: 1 (i.e., the RT comprises a variant of SEQ ID NO: 1) wherein one or more of the following amino acid substitutions are present: a Q to R at position 190, D to R at position 445, an E to K at position 509, and an E to K at position 588, wherein the positions correspond to positions in SEQ ID NO:1 . . . . In an embodiment, the RT has R at position 190, and optionally R at position 445 and/or K at position 509 and/or K at position 588. In an embodiment, the RT has R at position 445 and optionally has R at position 190 and/or K at position 509 and/or K at position 588. In an embodiment, the RT has K at position 509 and optionally R at position 190 and/or R at position 445 and/or K at position 588. In an embodiment, the RT has K at position 588 and optionally R at position 190 and/or R at position 445 and/or K at position 509. In an embodiment, the RT comprises an amino acid sequence having at least 90% identity with SEQ ID NO:1, wherein the amino acid sequence has an R at position 190, an R at position 445, a K at position 509, and a K at position 588, wherein the positions correspond to positions in SEQ ID NO:1. For example, the RT of SEQ ID NO:3 contains each of these amino acid substitutions.

In an embodiment, the RT comprises an amino acid sequence that has 10 or fewer, 9 or fewer, 8 or fewer, 7 or fewer, 6 or fewer, 5 or fewer, 4 or fewer, 3 or fewer, or 2 or fewer amino acid substitutions relative to SEQ ID NO:1. In an embodiment, the amino acid substitutions are conservative substitutions.

In some embodiments, the RT comprises an amino acid sequence that has at least 90% sequence identity to SEQ ID NO: 2 (e.g., at least 93%, at least 95%, at least 96%, at least 97%, at least 98%, or at least 99% sequence identity to SEQ ID NO: 2) but less than 100% sequence identity to the amino acid sequence of SEQ ID NO: 2. In an embodiment the RT comprises an amino acid sequence that has at least 95 sequence identity to SEQ ID NO:2. In an embodiment, the RT comprises an amino acid sequence that is identical to SEQ ID NO:2.

In some embodiments, the RT comprises an amino acid sequence that has at least 90% sequence identity (e.g., at least 93%, at least 95%, at least 96%, at least 97%, at least 98% or at least 99% sequence identity) to SEQ ID NO: 2 (i.e., the RT comprises a variant of SEQ ID NO: 2) wherein one or more of the following amino acid substitutions are present: a Q to R at position 190, an E to R at position 444, an E to K at position 507, and a D to K at position 586, wherein the positions correspond to positions in SEQ ID NO:2. In an embodiment, the RT has R at position 190, and optionally R at position 444 and/or K at position 507 and/or K at position 586. In an embodiment, the RT has R at position 444 and optionally has R at position 190 and/or K at position 507 and/or K at position 586. In an embodiment, the RT has K at position 507 and optionally R at position 190 and/or R at position 444 and/or K at position 586. In an embodiment, the RT has K at position 586 and optionally R at position 190 and/or R at position 444 and/or K at position 507. In an embodiment, the RT comprises and amino acid sequence having at least 90% identity with SEQ ID NO:2, wherein the amino acid sequence has an R at position 190, an R at position 444, a K at position 507, and a K at position 586, wherein the positions correspond to positions in SEQ ID NO:2. For example, the RT of SEQ ID NO:4 contains each of these amino acid substitutions

In an embodiment, the RT comprises an amino acid sequence that has 10 or fewer, 9 or fewer, 8 or fewer, 7 or fewer, 6 or fewer, 5 or fewer, 4 or fewer, 3 or fewer, or 2 or fewer amino acid substitutions relative to SEQ ID NO:2. In an embodiment, the amino acid substitutions are conservative substitutions.

Mutations to MMLV are well documented as described above, and such mutations can be incorporated into an RT amino acid sequence described above to produce a variant RT. Examples of such mutations that could be made to an amino acid sequence having at least 90% identity with SEQ ID NO:1 (e.g., at least 93%, at least 95%, at least 96%, at least 97%, at least 98% or at least 99% sequence identity to SEQ ID NO: 1) include any one or more of: K97S, 1102V, T115S, T115N, R120K, H181K, D186Q, F187W, R188P, R188T, V189P, V189Q, E193Q, L204M, L2051, P208E, P208Q, E210Q, G216W, G216D, A236L, 1249T, Q254T, R261M, K262R, T283S, A284V, A295G, T309Q, H317D, Q318E, E322K, L339I, K366Q, R367Q, V3691, T413V, D426F, L4421, G450V, L474V, V5181, A527T, F603M, V6081, H612K, and K618R. Similarly, examples of such mutations that could be made to an amino acid sequence having at least 90% identity with SEQ ID NO: 2 (e.g., at least 93%, at least 95%, at least 96%, at least 97%, at least 98% or at least 99% sequence identity to SEQ ID NO: 2), include any one or more of: K97S, 1102V, S115N, R120K, H181K, P186Q, F187W, R188P, R188T, A189P, A189Q, L204M, V2051, A208E, A208Q, K210Q, G216W, G216D, A236L, K254T, R261M, K262R, T283S, A284V, A295G, V309Q, H317D, Q318E, E322K, L339I, K366Q, R367Q, V3691, A413V, D426F, L4421, A449V, S473V, V5161, A525T, H601M, V6061, and H610K.

Any substitution of an amino acid in SEQ ID NO:1 or SEQ ID NO:2 (that is, in a “variant” of SEQ ID NO:1 or SEQ ID NO:2) can be a conservative substitution. The term “conservative substitution” means replacement of an amino acid in a polypeptide by one with similar characteristics; such substitutions are not likely to change the shape of the polypeptide chain, e.g., substituting one hydrophobic amino acid for another. For example, a non-polar amino acid (e.g., A, V, L, I, M, W, and F (and optionally C, G, and P)) may substitute for another non-polar amino acid, a polar amino acid (e.g., N, Q, S, T, and Y) may substitute for another polar amino acid (e.g., C, D, E, H, K, N, P. Q, R, S, and T), a positively charged amino acid (H, K, and R) may substitute for another positively charged amino acid, and a negatively charged amino acid (e.g., D and E) may substitute for another negatively charged amino acid. A substitute amino acid may be a natural amino acid (e.g., replacing another natural amino acid or a non-natural amino acid). A substitute amino acid may be a non-natural amino acid (e.g., replacing a natural amino acid or another non-natural amino acid). Examples of non-natural amino acids include norleucine, ornithine, norvaline, homoserine, and other amino acid analogs such as those described in Ellman et al. Meth, Enzym. 202:301-336 (1991).

As is described above, the RTs described herein have reverse transcriptase activity. In some embodiments, the RT has increased reverse transcriptase activity, and/or has improved tolerance to one or more reaction conditions such as high salt concentration, temperature, or the presence of reaction or sample components that inhibit reverse transcriptase activity, as compared to an RT known in the art.

Also provided are RTs that are fusion proteins comprising an exogenous amino acid sequence fused to an RT provided herein and described above. As used herein, the term “fusion protein” means a non-naturally occurring polypeptide containing two or more amino acid segments that are not joined in their naturally occurring states, e.g., a polymerase domain of an RT described here (where such polymerase domain has RT activity) and an amino acid sequence that is not joined to the polymerase domain in its naturally occurring state (an “exogenous” amino acid sequence). A fusion protein can be constructed for a variety of purposes, such as for ease of purification (a “purification tag,” e.g., poly-His, chitin binding domain, maltose binding protein, glutathione S-transferase (GST), alpha mating factor or SNAP-Tag® (New England Biolabs, Ipswich, MA)); for detection (e.g., a fluorescent protein for direct detection, an enzyme for indirect detection such as horse radish peroxidase); for protein translocation within a cell, tissue or organism (e.g., nuclear localization signal, mitochondrial targeting sequence, endoplasmic reticulum signal sequence, peroxisomal targeting signal, lysosomal targeting signal, organ-targeting signal); for protein interaction with other targets (e.g., DNA binding domain, which can be non-specific or specific); for chemical modification (e.g., to introduce a modification site), and the like. DNA binding domains have been shown to increase the processivity of other polymerases (see, e.g., US 2016/0160193); exemplary DNA binding domains include: Sso7d, BD007, BD023, BD009, BD062, BD093, BD109, BD006, and BD012. Accordingly, in some embodiments there is provided a fusion protein comprising the polymerase domain of an RT as defined herein (e.g., a polymerase domain having reverse transcriptase activity, comprising an amino acid sequence having at least 90% identity with SEQ ID NO: 1 or SEQ ID NO:2, or a polymerase domain having a reverse transcriptase activity comprising an amino acid sequence that is a portion of an amino acid sequence having at least 90% identity with SEQ ID NO:1 or SEQ ID NO:2) and any one or more of a purification tag; a detection moiety; a protein translocation sequence; a protein interaction sequence; and a sequence comprising a chemical modification or comprising a site that can be chemically modified. Thus, in some embodiments, the exogenous amino acid sequence comprises a purification tag, a detection moiety; a protein translocation sequence; a protein interaction sequence; and a sequence comprising a chemical modification or comprising a site that can be chemically modified.

An RT described herein can be joined with one or more of such domains at its N-terminus, C-terminus, and/or the middle portion located anywhere between the N- and C-terminus, or at more than one location. Segments of a fusion protein can optionally be separated by a linker. Polypeptide components of a fusion protein can be joined by one or more peptide bonds, disulfide linkages, and/or other covalent bonds. In an embodiment, the RT is a fusion protein comprising a polymerase domain that comprises an RT amino acid sequence described herein, and an exogenous amino acid sequence as defined herein. In some embodiments, there is provided a reverse transcriptase that comprises: (a) an amino acid sequence that is at least 90% identical to the amino acid sequence of SEQ ID NO:1 or SEQ ID NO: 2; and (b) a purification tag. In some embodiments, the reverse transcriptase fusion protein comprises (i) a polymerase domain comprising an amino acid sequence selected from: an amino acid sequence having at least 90% identity with SEQ ID NO:1; or an amino acid sequence having at least 90% identity with SEQ ID NO:2, or a portion of the selected amino acid sequence having reverse transcriptase activity; and (ii) an exogenous amino acid sequence.

For a particular fusion protein comprising a polymerase domain that is a segment of SEQ ID NO:1 or SEQ ID NO:2 (or a segment of a variant of SEQ ID NO:1 or SEQ ID NO:2 having an amino acid sequence with at least 90%, 92%, at least 93%, at least 95%, at least 96%, at least 97%, at least 98% or at least 99% sequence identity to SEQ ID NO:1 or SEQ ID NO:2, respectively), it is understood that the % sequence identity is determined with respect to the number of amino acids in the polymerase domain (and not with respect to the number of amino acids in the full length SEQ ID NO:1 or SEQ ID NO:2 unless the polymerase domain corresponds to the full length of SEQ ID NO:1 or SEQ ID NO:2 or the variant thereof).

The RT fusion proteins described herein have reverse transcriptase activity. In some embodiments, the RT fusion protein has increased reverse transcriptase activity, and/or has improved tolerance to one or more reaction conditions such as high salt concentration, temperature, or the presence of reaction or sample components that inhibit reverse transcriptase activity, as compared to an RT known in the art and/or as compared to a fusion protein comprising an RT known in the art (e.g., an RT fusion protein containing an exogenous amino acid sequence of equivalent function).

Also provided by the present disclosure are compositions including an RT described herein. Such a composition can include one or more RTs and one or more substances selected for purposes such as storage stability (including a substance such as a solid support, gel, or solution); detection of presence, concentration, or activity of the reverse transcriptase; and/or for performing a method using the reverse transcriptase (e.g., providing the RT with other components for polynucleotide extension of a target nucleic acid (e.g., RNA template or DNA template)), and/or template switching reaction, referenced in some instances as a reaction mixture).

A composition can therefore contain components for polynucleotide extension (e.g., extension of nucleic acid target), such as dNTPs. Compositions containing dNTPs can include one, two, three or all four of dATP, dTTP, dGTP and dCTP, and can include one or more modified dNTPs, such as forms that are resistant to, or susceptible, to a particular enzymatic or chemical conversion (e.g., deaminase-resistant), or that are detectable. Examples of modified dNTPs include alpha-phosphorothioate dNTPs, dUTP, dITP, labeled dNTPs such as, e.g., fluorescein- or cyanin-dye family dNTPs, radiolabeled dNTPs.

An RT composition can include any of (including one or more of) a buffering agent (e.g., HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, triethanolamine); an excipient; a salt, such as a cationic salt generally required for polynucleotide extension activity of an RT (e.g., NaCl, MnCl₂, MgCl₂, MgSO₄, CaCl₂)); a protein (e.g., albumin; an enzyme, such as a uracil DNA glycosidase (UDG), a polymerase such as Taq polymerase, e.g., for performing qPCR procedures; a nucleic acid binding protein, e.g., Gp32), a dye (e.g., for detecting the presence, concentration or activity of the RT, including detecting cDNA molecules generated such as in qPCR methods); a stabilizer; an inhibitor (e.g., RNase inhibitor, such as human placental RNase inhibitors, porcine liver RNase inhibitors, mouse RNA inhibitor, 2′-cytidine monophosphate free acid (2′-CMP), aluminon, adenosine 5′-pyrophosphate); a detergent (for example, ionic, non-ionic, and/or zwitterionic detergents, a poloxamer); a reducing agent (e.g., dithiothreitol, beta mercaptoethanol); an polynucleotide such as one or more oligonucleotide (e.g., template switching oligos), primers and/or control polynucleotide; a cell (e.g., intact, digested, or any cell-free extract); a biological sample; an aptamer (e.g., for controlling the activity of an RT); a crowding agent (e.g., polyethylene glycol, e.g., PEG6000); a sugar (e.g., a mono, di, tri, tetra, or higher saccharide); a starch; cellulose; a glass-forming agent (e.g., for lyophilization); a lipid; an oil; aqueous media; a support (e.g., a matrix such as a bead, filter paper, slide) and/or (non-naturally occurring) combinations thereof. Combinations can include, for example, two or more of the listed components (e.g., a salt and a buffering agent) or a plurality of a single listed component (e.g., two different salts or two different sugars). Those skilled in the art will be able to identify additional components for their use of the disclosed RT in a composition (or method or kit, as described below).

Thus, in an embodiment, a composition includes an RT described herein. In various embodiments, the composition can include one or more of: dNTPs, a buffering agent, an oligonucleotide (e.g., a primer, a template switching oligonucleotide), a cationic salt (e.g., a divalent salt and optionally a monovalent salt), a DNA polymerase, an RNA template, a DNA template, a DNA binding protein (e.g., T4 GP32, for example when performing a template switching reaction) and an aptamer. In an embodiment, the RT is associated with (e.g., covalently or non-covalently attached to) a solid support. In an embodiment, the composition is lyophilized, dried, and/or frozen.

The RTs described herein can be used for any purpose in which their activity is necessary or desired, for example to copy RNA into DNA and/or copy DNA into DNA via the DNA polymerase activity of an RT; to employ the template switching activity for any purpose; or both. The RTs described herein can have reverse transcriptase activity; template switching activity; or both reverse transcriptase and template switching activity. In an embodiment, the RT has reverse transcriptase activity. In an embodiment, the RT has reverse transcriptase and template switching activity. Accordingly, the RTs described herein can be useful, e.g., for first strand cDNA synthesis, second strand cDNA synthesis, RT-PCR, RT-qPCR, RNA-seq library preparation, 5′RACE. When performing some methods, additional enzymes can be necessary or beneficial for high performance. For example, Examples 6-9 describe RT-qPCR methods that employ Taq polymerase (Luna® Universal Probe qPCR Master Mix) for the quantitative PCR.

Accordingly, provided herein are methods, which involve incubating a reaction mixture comprising (i) a reverse transcriptase described herein, (ii) a primer, (ii) dNTPs, and (iii) a target nucleic acid, under conditions suitable for copying of the template (polynucleotide extension of a primer, resulting in copying of the nucleic acid template to produce a copied DNA product).

Also provided herein are methods for template switching employing an RT described herein. As background, conventional cDNA construction strategies sometimes underrepresent the 5′ end sequences of the mRNA. A known approach to solving this problem is template switching using a chimeric DNA: RNA oligo and a reverse transcriptase having template switching activity to improve 5′ transcript coverage in a sequence-independent manner and thereby obtain better coverage of the sequences present in the mRNA library. Thus, a described RT can be used for this among other template switching applications. In an embodiment, an RT described herein can be combined with a DNA binding protein such as T4 Gene 32 Protein in a template switching reaction (see, e.g., Example 10).

The RTs described herein are useful for polynucleotide extension of nucleic acid templates using a primer, also referenced herein copying a target nucleic acid to generate copied DNA. As generally used herein, the term “nucleic acid” means a polymeric form of nucleotides of any length, such as deoxyribonucleotides or ribonucleotides, or analogs thereof. For example, a nucleic acid can be DNA, RNA or the DNA product of RNA subjected to reverse transcription. Non-limiting examples of nucleic acids include coding or non-coding regions of a gene or gene fragment, loci (locus) defined from linkage analysis, exons, introns, messenger RNA (mRNA), transfer RNA, ribosomal RNA, ribozymes, cDNA, recombinant nucleic acids, branched nucleic acids, plasmids, vectors, isolated DNA of any sequence, isolated RNA of any sequence, nucleic acid probes, and primers. Other examples of nucleic acids include, without limitation, cDNA, aptamers, and peptide nucleic acids. Nucleic acid can contain modified nucleotides, such as methylated nucleotides and nucleotide analogs (“analogous” forms of purines and pyrimidines are well known in the art). If present, modifications to the nucleotide structure can be imparted before or after assembly of the polymer. Nucleic acid can be a single-stranded, double-stranded, partially single-stranded, or partially double-stranded DNA or RNA, depending on the application. As used herein, the term “polynucleotide extension” means the synthesis of DNA catalyzed by an RT resulting in polymerization of individual nucleoside triphosphates using a primer as a point of initiation. A primer is hybridized to a target nucleic acid (RNA or DNA) to form a primer-template complex. The primer-template complex is contacted with the RT and nucleoside triphosphates (dNTPs) in a suitable environment to permit the addition of nucleotides to the 3′ end of the primer, thereby producing a copied DNA product complementary to at least a portion of the target nucleic acid.

As used herein, the term “primer” means an oligonucleotide that is capable of, upon forming a duplex with a polynucleotide template, acting as a point of initiation of DNA synthesis and being extended from its 3′ end along the template so that an extended duplex is formed. The sequence of nucleotides added during the extension process is determined by the sequence of the template polynucleotide. Primers are of a length compatible with their use in synthesis of primer extension products, and can be in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18 to 40, 20 to 35, 21 to 30 nucleotides long, and any length between the stated ranges. Primers are usually single stranded. Primers have a 3′ hydroxyl.

One or more sequence-specific primers can be used for RT-PCR (e.g., quantitative RT-PCR) and other similar analyses. Primers containing oligo-dT and random primers can be used when making a cDNA library, e.g., to be sequenced, used for gene expression analysis (e.g., RACE). Template switching oligonucleotides (described above) can be used for template switching applications such as adding tags to polynucleotide strands. In conjunction with a template switching oligonucleotide, cDNA is synthesized with a known sequence of choice attached to the 3′ end. The resulting cDNA can be amplified by PCR or serve as template for, e.g., 5′ RACE (rapid amplification of cDNA ends) or second strand cDNA synthesis.

A primer used for polynucleotide extension using an RT can contain a feature (e.g., chemical moiety, sequence, modified nucleotide, etc.) for the detection or immobilization of the primer so long as such feature(s) do not destroy the ability of the primer to act as a point of initiation of DNA synthesis. For example, primers can contain an additional nucleic acid sequence at the 5′ end that does not hybridize to the target nucleic acid, but that facilitates cloning or sequencing of the amplified product. A primer can include conventional nucleotides, unconventional nucleotides (e.g., ribonucleotides or labeled nucleotides), nucleotide analogs, and mixtures thereof, as suitable for a particular application. A primer can have a detectable tag (e.g., a fluorescent tag).

Conditions suitable for polynucleotide extension are known in the art (see, e.g., Sambrook et al., supra. See also Ausubel et al., Short Protocols in Molecular Biology (4th ed., John Wiley & Sons 1999). In general, a reaction mixture for carrying out polynucleotide extension using an RT may include a buffering agent, dNTPs, a divalent cation (e.g., Mg²⁺, Mn²⁺, Co²⁺, Cd²⁺), a monovalent cation (e.g., a salt such as KCl and/or NaCl), one or more primers, and optionally can include an antibody, antibody-like molecule, an aptamer, or other entity to inhibit the RT or another reaction component under selected conditions (such as temperature or salt concentration). The design of aptamers is well known (see, e.g., Byun J. Life (Basel). 2021 Feb. 28; 11 (3): 193). A reaction mixture can have a pH range of about 7.5-8.8 and polynucleotide extension can be carried out at a temperature range of about 40° C.-65° C. as described herein below. The RTs described herein are active in a greater temperature range, and particularly at higher temperatures than some commercially available RTs (see, e.g., Example 11).

Target nucleic acids, which can be RNA templates or DNA templates, are substrates for the RTs described herein. An RNA template used herein can be any type of RNA template, e.g., total RNA, polyA+ RNA, capped RNA, enriched RNA. An RNA template can be from any source, e.g., bacteria, mammals, an in vitro transcription reaction, etc., processes for the making of which are known. For example, Examples 2-4 describe use of in vitro transcribed RNA templates; Examples 5 and 6 describe use of cultured cell derived RNA. The RNA template can contain RNA molecules that are at least 1 kb in length, e.g., at least 3 kb, at least 5 kb, at least 8 kb, at least 10 kb, at least 16 kb, at least 20 kb. For example, Example 4 describes using polyA RNA templates of 1, 4, and 8 kb for first strand cDNA synthesis, and Example 9 describes using 16 kb RNA template in a two-step RT-qPCR method. The starting amount of RNA template is determined by the particular application and can be in the range of, for example, 1 μg to 100 ng (e.g., 5 pg to 50 ng) for one-step RT-PCR, or in the range of, for example, 0.5 pg to 5 μg (e.g., 1 pg to 1 μg) for two-step RT-PCR. For example, Example 5 describes using 5 pg-50 ng of Jurkat RNA for performing one step RT-qPCR; Example 7 describes using 1 pg-1 μg for two step RT-PCR; and other starting amounts of RNA templates are described in further examples as well as in published literature. A DNA template used herein can be any type of DNA template, e.g., cDNA resulting from first strand synthesis, e.g., by an RT described herein, double-stranded DNA, hybrid.

The RTs described herein can be used in generating copied DNA products from nucleic acids obtained from diverse sources, such as people, animals and plants, environments (e.g., soils, waters, vehicles, homes, hospitals, airports), food products, forensic and archaeological materials. Thus, as used herein, the term “sample” means a natural or man-made substance suspected of containing a target nucleic acid, such as a biological fluid, cell, tissue, or fraction thereof, food or environmental substance that can contain or be contaminated by a target nucleic acid. A sample can be derived from a prokaryote or eukaryote and therefore can include cells from, for example, animals, plants, or fungi as well as viruses. Accordingly, a sample includes a specimen obtained from one or more individuals or can be derived from such a specimen. For example, a sample can be a tissue section obtained by biopsy, or cells that are placed in or adapted to tissue culture. Exemplary samples include biological specimens such a cheek swab, nasopharyngeal swab, throat swab, nasopharynx flush through, amniotic fluid, skin biopsy, organ biopsy, tumor biopsy, blood, urine, saliva, semen, sputum, cerebral spinal fluid, tears, mucus, and the like. A sample can be further fractionated, if desired, to a fraction containing particular cell types. For example, a blood sample can be fractionated into serum or into fractions containing particular types of blood cells. If desired, a sample can be a combination of samples from an individual such as a combination of a tissue and fluid, or a combination of samples from more than one individual (e.g., pooled samples, maternal sample containing fetal nucleic acid). Prior to analysis, a sample can be processed to preserve the integrity of nucleic acid targets. Such methods include the use of appropriate buffers and/or inhibitors, including nuclease, protease, and phosphatase inhibitors, which preserve or minimize changes in the molecules in the sample, including tissue fixatives (e.g., in the case of FFPE preserved tissues).

A sample can contain more than one target nucleic acid, either naturally or due to combination prior to analysis. Thus, the methods employing an RT described herein can be used to detect more than one target nucleic acid simultaneously (e.g., at least two, more than two, at least five, more than five, at least ten, or more than ten). For example, Example 8 describes a 5-plex two-step RT-qPCR method. A sample can contain a substance that can inhibit reverse transcriptase activity of some RTs under reaction conditions in which an RT described herein has activity, such as an amount of salt, chemical, dyes, lysis reagents, environment and animal sample components and other substances.

In some embodiments, the method further involves quantifying the amount of the copies of the target nucleic acid.

In some embodiments, the RT is tolerant of one or more of a high salt condition, a chemical, a dye, a lysis reagent, an environmental component, an animal sample component, or other sample contaminant or reaction component, as is described herein. In some embodiments, the RT is salt-tolerant.

In some embodiments, the RT is in a composition (e.g., reaction mixture) comprising one or more salts. For example, the salt(s) may be selected from potassium (e.g. KCl), magnesium (e.g. MgCl₂), lithium (e.g. LiCl), and sodium (e.g. NaCl, Na citrate, Na acetate, or Na heparin). For example, in some embodiments, the RT is in a composition (e.g., reaction mixture) comprising any one or more salt selected from potassium (e.g. KCl) at a concentration of up to 400 mM, optionally 20 mM to 400 mM, 40 mM to 300 mM, 150 mM to 250 mM (e.g. 250 mM KCl); magnesium (e.g. MgCl₂) at a concentration of up to 12 mM, optionally 1 mM to 12 mM, 1.5 mM to 6 mM, or 8 mM to 12 mM; lithium (e.g. LiCl) at a concentration of up to 250 mM, optionally 150-250 mM; NaCl at a concentration up to 250 mM, optionally 150-250 mM; Na citrate at a concentration of up to 50 mM, optionally 25-50 mM; Na acetate at a concentration of up to 175 mM, optionally 125-175 mM; and Na heparin at a concentration of up to 0.01 mM, optionally 0.0025 to 0.01 mM. As demonstrated herein, an exemplary RT of the invention has reverse transcriptase activity in the presence of any of these salt concentrations.

In some embodiments, the RT composition (e.g., reaction mixture) comprises a buffer. For example, the composition (e.g., reaction mixture) is at a pH in the range of 7.5 to 8.5 (e.g. pH 8.5). An RT of the invention has reverse transcriptase activity within this pH range.

In some embodiments, the reaction mixture can be incubated at a temperature in the range of 35° C. to 65° C., e.g., at a temperature of 42° C. to 65° C., optionally at a temperature of about 55° C. or in the range of 55° C. to 65° C. In some embodiments, the reaction mix can be incubated at a temperature of at least 42° C., 45° C., 48° C., 51° C., 54° C., 57° C., 60° C., or 65° C.; or at a temperature in the range of 42° C. to 45° C., 48° C. to 51° C., 51° C. to 54° C., 54° C. to 57° C., 57° C. to 60° C., or 60° C. to 65° C. Example 1 demonstrates that exemplary RTs described herein have RT activity at 58° C. and 62° C. Example 11 describes the activity of an exemplary RT at temperatures between 42° C. to 65° C.

As is described herein, an RT disclosed herein is capable of synthesizing high yields of cDNA product from an RNA template (such as an RNA template of a size of at least 1 kb, at least 4 kb, at least 8 kb, at least 16 kb) in short reaction times, such as a time period selected from 10 minutes, 9 minutes, 8 minutes, less than 10 minutes, less than 9 minutes, and less than 8 minutes.

In some embodiments, the RT has reverse transcriptase activity in a reaction mixture comprising KCl at a concentration of 40 mM to 300 mM (e.g. 40-120 mM) and MgCl₂ at a concentration of 1.5 mM to 6 mM; optionally wherein the pH is in the range of 7.5 to 8.5 and/or the reaction temperature is about 55° C.

For example, a general-purpose reaction mixture suitable for generating long cDNA (e.g., 8-16 kb) using an RT of the invention comprises KCl at a concentration of about 120 mM and MgCl₂ at a concentration of about 3 mM; and optionally further comprises about 0.02% Tween-20 and/or has a pH of 8.5. In some specific embodiments, an RT as disclosed herein is capable of synthesizing high yields of a long (e.g. about 8 kb or about 16 kb) cDNA product from an RNA template within 10 minutes in this general purpose reaction mixture at about 55° C.

In some embodiments, the RT composition (e.g., reaction mixture) comprises one or more commonly used chemical inhibitors, dyes, or cell lysis substances; for example, any one or more of Tween (e.g. Tween 20), ethanol, isopropanol, DMSO, SDS, urea, guanidine iso thiocyanate, DTT, hydrogen peroxide, SYBR® Green II, SYBR Gold, formalin, Solulyse reagent, BugBuster® reagent, and Luna Cell Ready reagent. For example, the RT composition (e.g., reaction mixture) may comprise Tween 20 at a concentration of up to 0.04 mM (e.g. 0.01-0.04 mM), ETOH at a concentration of up to 0.1 mM (e.g. 0.1 mM), isopropanol at a concentration of up to 0.1 mM (e.g. 0.1 mM), DMSO at a concentration of up to 0.15 mM (e.g. 0.05-0.15 mM), SDS at a concentration of up to 5e-05 mM (e.g. 1e-05 to 5e-05 mM), Urea at a concentration of up to 2000 mM (e.g. 1000-2000 mM), guanidine iso thiocyanate at a concentration of up to 75 mM (e.g. 75 mM), DTT at a concentration of up to 100 mM (e.g. 10-100 mM, and/or hydrogen peroxide at a concentration of up to 50 mM (e.g. 25-50 mM). For example, the RT composition (e.g., reaction mixture) may comprise SYBR Green II dye at a concentration of up to 50 mM (e.g. 10-50 mM), SYBR Gold dye at a concentration of up to 20 mM (e.g. 10-20 mM), formalin at a concentration of up to 0.001 mM (e.g. 0.0002-0.001 mM), Solulyse reagent at a concentration of up to 0.2 mM (e.g. 0.1-0.2 mM), BugBuster reagent at a concentration of up to 0.02 mM (e.g. 0.01-0.02 mM), and/or Luna Cell Ready reagent at a concentration of up to 0.02 mM (e.g. 0.1-0.2 mM). As demonstrated herein, an exemplary RT of the invention has reverse transcriptase activity in the presence of these components at the recited concentrations/concentration ranges.

In some embodiments, the RT composition (e.g., reaction mixture) comprises one or more environmental or animal sample components, optionally tannic acid, hemin, hematin, humic acid, melanin, hemoglobin, and myoglobin. For example, the RT composition (e.g., reaction mixture) may comprise tannic acid at a concentration of up to 0.01 mM (e.g. 2e-05 to 0.01 mM), hemin at a concentration of up to 10 mM (e.g. 5-10 mM), hematin at a concentration of up to 20 mM (e.g. 2-20 mM), humic acid at a concentration of up to 1 mM (e.g. 0.5-1 mM), melanin at a concentration of up to 1 mM (e.g. 0.5-1 mM), hemoglobin at a concentration of up to 5 mM (e.g. 1-5 mM), and/or myoglobin at a concentration of up to 5 mM (e.g. 1-5 mM). As demonstrated herein, an exemplary RT of the invention has reverse transcriptase activity in the presence of these components at the recited concentrations/concentration ranges.

Also provided by the present disclosure are kits for using an RT described herein. A kit can include one or more RTs described herein together with one or more other components useful for carrying out a method involving RNA polynucleotide extension, and/or DNA polynucleotide extension, and/or template switching.

A kit can therefore contain components for polynucleotide extension (e.g., amplification of a nucleic acid target), and/or template switching (e.g., amplification of a nucleic acid targets with addition of sequences via a template switching oligonucleotide such as for sequencing library preparation), such as dNTPs. A kit containing dNTPs can include one, two, three of all four of dATP, dTTP, dGTP and dCTP, and can include one or more modified dNTPs, such as forms that are resistant to, or susceptible, to a particular enzymatic or chemical conversion, or that are detectable. Examples of modified dNTPs include alpha-phosphorothioate dNTPs, dUTP, dITP, labeled dNTPs such as, e.g., fluorescein- or cyanin-dye family dNTPs. A kit can include radiolabeled dNTPs, such as ³HdTTP.

A kit can include a reaction mixture in any convenient form, such as in solution, concentrated form, dried form, disposed in, on, or within a solid support (e.g., a tube, plate, pellet, membrane, bead). In an embodiment, one or more components is associated with (e.g., covalently or non-covalently attached to) a solid support. Such a reaction mixture can contain components useful for enabling use of the RT in a particular assay format, e.g., to promote a particular aspect of the RT enzymatic activity, a molecular interaction, a stability profile, and other desirable properties. Accordingly, reaction mixture can contain one or more salts, detergents (ionic, non-ionic, zwitterionic), poloxamers, preservatives, inhibitors of unwanted activities, crowding agents, reducing agents (e.g., DTT), catalysts, dyes, and other substances. In an embodiment, the reaction mixture is suitable for receiving and amplifying a nucleic acid template in the presence of the RT and one or more primers.

In an embodiment, the RT is in a form selected from: dried, lyophilized, and in solution, wherein the solution is optionally glycerol-free. In some embodiments, a kit includes one or more oligonucleotides that bind to a predetermined nucleic acid template. In the case of oligonucleotide primers, the primers can be for an RNA template or DNA template for polynucleotide extension. In some embodiments, the kit includes one or more oligo dT primers. In some embodiments, a kit does not include primers or includes a limited number of primers, in instances where the kit user provides primers appropriate for their selected target. In some embodiments, a kit includes a control primer (e.g., rActin control). In some embodiments, a kit includes target-specific primers (e.g., to detect a pathogen). In some embodiments, a kit includes one or more of an oligo dT primer, a random primer, and a target specific primer such as a gene-specific primer. If desired, a primer can be exonuclease-resistant and/or chemically modified.

A kit can include an aptamer, e.g., for binding to an RT to control the conditions under which the RT has activity (e.g., to reduce off-target binding/amplification), for binding to another component in the kit (e.g., another enzyme such as a DNA polymerase), to control the activity of a sample constituent (e.g., to inhibitor RNAse).

A kit can include a probe, e.g., for detecting copied DNA, such as when performing qPCR. A kit can also include instructions for practicing a desired method (e.g., amplifying a target nucleic acid, detecting a target nucleic acid, sequencing library preparation) via any communication means. For example, the instructions can be printed (e.g., on paper or plastic), and/or electronic (e.g., provided on a device such as a portable drive, or remotely accessible such as on a web application, phone application, video, or voice transmission), and/or via demonstration.

A kit can include one or more other enzymes, as suitable for a particular purpose. For example, a kit can contain a UDG enzyme for reducing carryover contamination in PCR assays, which can lead to false positives. A UDG can be employed in a workflow to degrade amplified DNA that contains Us, leaving native target nucleic acids intact. Thus, UDG treatment is often performed as a treatment prior to DNA amplification. A kit can include a DNA polymerase (e.g., Taq polymerase) for amplification of target nucleic acids, e.g., for DNA target amplification in RT-PCR.

A kit can include a control, such as a control polynucleotide, which can be a plasmid or linear nucleic acid, depending on the application.

Components of a kit can be provided in a single container or compartment (e.g., for a single step use) or multiple containers or compartments (e.g., for combining, for sequential use, for parallel use, or another desired workflow). A kit can include a sample collection container, which optionally can contain a reagent, e.g., for stabilizing the sample (e.g., a poloxamer) or preparing it for assay.

A kit can contain RT described herein and a reaction mixture. The reaction mixture can contain a buffering agent and can include a cationic salt as well as other components described above. For example, a kit can further include one or more components selected from dNTPs, a primer, an aptamer, a detergent.

A kit can contain an RT that is thermostable at a particular temperature, as described herein above such as in Example 11, e.g., to enable use of reaction mixtures that will contain an RT during a heating process (e.g., heat lysis of cells, heat denaturation of nucleic acids).

### Exemplary Embodiments

Embodiment 1. An engineered reverse transcriptase comprising: an amino acid sequence having at least 90% identity with SEQ ID NO:1; or an amino acid sequence having at least 90% identity with SEQ ID NO:2.

Embodiment 2. The reverse transcriptase of embodiment 1, wherein amino acid sequence has at least 95% sequence identity with SEQ ID NO:1 or SEQ ID NO:2.

Embodiment 3. The reverse transcriptase of any prior embodiment, wherein the amino acid sequence is identical to SEQ ID NO:1 or SEQ ID NO:2.

Embodiment 4. A reverse transcriptase of embodiment 1, comprising: an amino acid sequence having at least 90% identity with SEQ ID NO:1, wherein the amino acid sequence has the following amino acid substitutions: an R at position 191, an R at position 446, a K at position 510, and a K at position 589, wherein the positions correspond to positions in SEQ ID NO:1 preceded by an M.

Embodiment 5. A reverse transcriptase of embodiment 1, comprising: an amino acid sequence having at least 90% identity with SEQ ID NO:2, wherein the amino acid sequence has the following amino acid substitutions: a Q to R at position 191, an E to R at position 445, an E to K at position 508, and a D to K at position 587, wherein the positions correspond to positions in SEQ ID NO:2 preceded by an M.

Embodiment 6. The reverse transcriptase of any prior embodiment, wherein the reverse transcriptase is a fusion protein comprising: (i) a polymerase domain comprising the amino acid sequence; and (ii) an exogenous sequence.

Embodiment 7. The reverse transcriptase of embodiment 6, wherein the exogenous sequence comprises a purification tag.

Embodiment 8. A composition comprising a reverse transcriptase of any prior embodiment.

Embodiment 9. The composition of embodiment 8, further comprising one or more of: dNTPs, a buffering agent, an oligonucleotide, and an aptamer.

Embodiment 10. The composition of embodiment 8, wherein reverse transcriptase is associated with a solid support.

Embodiment 11. The composition of embodiment 8, wherein the composition is in a form selected from lyophilized, dried, and in solution.

Embodiment 12. A kit comprising: (i) a reverse transcriptase of any of embodiments 1-7; and (ii) a reaction mixture.

Embodiment 13. The kit of embodiment 12, wherein the reaction mixture comprises a buffering agent.

Embodiment 14. The kit of any prior embodiment, wherein the reaction mixture further comprises a cationic salt.

Embodiment 15. The kit of any prior embodiment, further comprising one or more components selected from dNTPs, a primer, an aptamer, a detergent, a template switching oligonucleotide.

Embodiment 16. The kit of any prior embodiment, wherein the reverse transcriptase is in a form selected from lyophilized, dried, and in solution.

Embodiment 17. The kit of any prior embodiment, wherein one or more components is associated with a solid support.

Embodiment 18. A method comprising: incubating a reaction mixture comprising (i) a reverse transcriptase of any of embodiments 1-7, (ii) a primer, (ii) a target nucleic acid, and (iii) dNTPs, under conditions suitable for polynucleotide extension of the target nucleic acid to generate copied DNA.

Embodiment 19. The method of any embodiment herein, wherein the incubating is done at a temperature of between 55° C.-65° C.

Embodiment 20. The method of any embodiment herein, wherein the primer is selected from an oligo (dT) primer, a random primer, a gene-specific primer.

Embodiment 21. The method of any embodiment herein, wherein the target nucleic acid is an RNA template.

Embodiment 22. The method of any of embodiment herein, wherein the target nucleic acid is a DNA template.

Embodiment 23. The method of embodiment 18, wherein the target nucleic acid is an RNA template, and further comprising incubating the generated copied DNA with an enzyme to generate second copied DNA, optionally wherein the enzyme is a reverse transcriptase of any of embodiments 1-7.

Embodiment 24. The method of embodiment 18, wherein generation of copied DNA is detected using a quantitative method.

Embodiment 25. The method of embodiment 21, wherein the RNA template is of a size of at least about 1 kb, and full length copied DNA is generated within a time selected from: 10 minutes, 9 minutes, and 8 minutes.

Embodiment 26. A reverse transcriptase comprising: an amino acid sequence having at least 90% identity with SEQ ID NO:1; or an amino acid sequence having at least 90% identity with SEQ ID NO:2.

Embodiment 27. The reverse transcriptase of embodiment 26, wherein the amino acid sequence has at least 95% sequence identity with SEQ ID NO:1 or SEQ ID NO:2.

Embodiment 28. The reverse transcriptase of Embodiment 26 or 27, wherein the amino acid sequence is identical to SEQ ID NO:1 or SEQ ID NO:2.

Embodiment 29. The reverse transcriptase of embodiment 26, comprising:
an amino acid sequence having at least 90% identity with SEQ ID NO: 1, wherein the amino acid sequence has one or more of the following amino acid substitutions:
   an R at position 190,
   an R at position 445,
   a K at position 509, and
   a K at position 588,
   wherein the positions correspond to positions in SEQ ID NO:1.

Embodiment 30. The reverse transcriptase of embodiment 29, wherein the amino acid sequence contains
an R at position 190,
an R at position 445,
a K at position 509, and
a K at position 588,
wherein the positions correspond to positions in SEQ ID NO:1

Embodiment 31. The reverse transcriptase of embodiment 26, comprising:
an amino acid sequence having at least 90% identity with SEQ ID NO:2, wherein the amino acid sequence has one or more of the following amino acid substitutions:
   an R at position 190,
   an R at position 444,
   a K at position 507, and
   a K at position 586,
   wherein the positions correspond to positions in SEQ ID NO:2.

Embodiment 32. The reverse transcriptase of embodiment 31, wherein the amino acid sequence contains
an R at position 190,
an R at position 444,
a K at position 507, and
a K at position 586,
wherein the positions correspond to positions in SEQ ID NO:2.

Embodiment 33. The reverse transcriptase of any preceding embodiment, wherein the reverse transcriptase is a fusion protein comprising: (i) a polymerase domain comprising the amino acid sequence; and (ii) an exogenous amino acid sequence.

Embodiment 34. A reverse transcriptase fusion protein comprising:
(i) a polymerase domain having reverse transcriptase activity, comprising an amino acid sequence that is a portion of:
an amino acid sequence having at least 90% identity with SEQ ID NO:1; and
an amino acid sequence having at least 90% identity with SEQ ID NO:2; and (ii) an exogenous amino acid sequence.

Embodiment 35. The reverse transcriptase of embodiment 33 or 34, wherein the exogenous amino acid sequence comprises a purification tag.

Embodiment 36. A composition comprising a reverse transcriptase of any preceding embodiment.

Embodiment 37. The composition of embodiment 36, further comprising one or more of: dNTPs, a buffering agent, an oligonucleotide, an aptamer, a template switching oligonucleotide, a DNA polymerase, an RNA template and a DNA template.

Embodiment 38. The composition of embodiment 36, wherein reverse transcriptase is associated with a solid support.

Embodiment 39. The composition of embodiment 36, wherein the composition is in a form selected from lyophilized, dried, and in solution.

Embodiment 40. A kit comprising:
(i) a reverse transcriptase of any of embodiments 26-35; and
(ii) a component selected from a buffering agent, a cationic salt, dNTPs, a primer, an aptamer, a detergent, a template switching oligonucleotide, and nucleic acid binding protein, wherein the nucleic acid binding protein is optionally T4 Gene Protein 32.

Embodiment 41. The kit of embodiment 40, wherein the reverse transcriptase is in a form selected from lyophilized, dried, and in solution.

Embodiment 42. The kit of embodiment 40 or embodiment 41, wherein one or more components is associated with a solid support.

Embodiment 43. A method comprising:
incubating a reaction mixture comprising
   (i) a reverse transcriptase of any of embodiments 26-35,
   (ii) a primer,
   (ii) a target nucleic acid, and
   (iii) dNTPs,
under conditions suitable for the reverse transcriptase to copy the target nucleic acid to generate copied DNA.

Embodiment 44. The method of embodiment 43, wherein the incubating is done at a temperature of between 55° C.-65° C.

Embodiment 45. The method of any embodiment herein, wherein the primer is selected from an oligo (dT) primer, a random primer, a target-specific primer.

Embodiment 46. The method of any embodiment herein, wherein the target nucleic acid is RNA.

Embodiment 47. The method of any of embodiment herein, wherein the target nucleic acid is a DNA.

Embodiment 48. The method of embodiment 43, wherein the target nucleic acid is RNA, and further comprising incubating the generated copied DNA with an enzyme to generate second copied DNA, optionally wherein the enzyme is a reverse transcriptase of any of embodiments 1-7.

Embodiment 49. The method of embodiment 43, further comprising quantifying the amount of the copies of the target nucleic acid.

Embodiment 50. The method of embodiment 46, wherein the RNA template is of a size of at least about 1 kb, and full length copied DNA is generated within a time period selected from 10 minutes, 9 minutes, 8 minutes, less than 10 minutes, less than 9 minutes, and less than 8 minutes.

Embodiment 51. The method of embodiment 50, wherein the full length copied DNA is generated within 9 minutes.

Embodiment 52. The method of embodiment 50, wherein the full length copied DNA is generated within 8 minutes.

Embodiment 53. The RT of embodiment 30, wherein the RT comprises SEQ ID NO:3.

Embodiment 54. The RT of Embodiment 32, wherein the RT comprises SEQ ID NO:4.

The skilled artisan will understand that the figures, described above, and examples, described below, are for illustration purposes only. Neither the figures nor the examples are intended to limit the scope of the disclosed teachings in any way.

### Example 1. Activity of Engineered RTs

Engineered reverse transcriptases ERT1 (SEQ ID NO:1), ERT2 (SEQ ID NO:2), an exemplary variant of ERT1 (ERT3 (SEQ ID NO:3)), and an exemplary variant of ERT2 (ERT4 (SEQ ID NO: 4)) were expressed as fusion proteins containing the following sequence at their N termini: MGKIEESKHHHHHHGS (SEQ ID NO:8). For clarity, this sequence was used for ease of purification and can be omitted or substituted for an alternative purification tag (many purification tags are described herein above and/or described in the literature). The fusion proteins are used in all experiments that follow.

Recombinantly expressed and purified engineered reverse transcriptase fusion proteins ERT1 and ERT2 and their respective variants, ERT3 and ERT4, efficiently synthesize cDNA product under stringent reaction conditions. A commercially available reverse transcriptase (CAV RT1) (M3025, New England Biolabs, Inc.) and ERT1-4 were expressed, purified, and assayed for cDNA synthesis via extension of a fluorescently labeled DNA primer annealed to an in vitro transcribed 450 bp RNA template. Assays were performed in 50 mM Tris-HCl [pH 8.5], 250 mM KCl (high salt condition), 3 mM MgCl₂, and 0.02% Tween-20. Briefly, reactions containing 100 nM primer: template were initiated with 80 nM enzyme, incubated at 58° C. for 10 minutes and stopped by heat inactivation at 95° C. for 5 minutes. Results were visualized by capillary electrophoresis, analyzed by PeakScanner software and full-length product was reported relative to total integrated signal. Under these stringent conditions, ERT1-4 all displayed significantly more cDNA synthesis than CAV RT1.

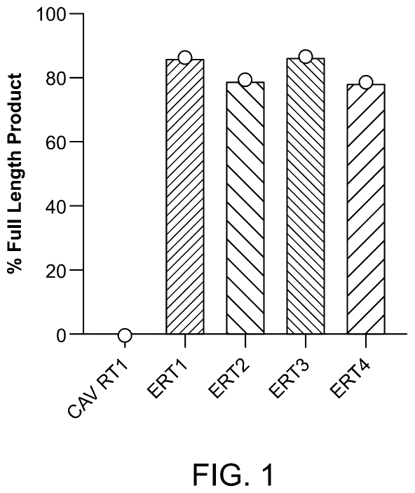

As shown in Table 1 and FIG. 1, RT activity as indicated by generation of copied DNA was consistent in standard KCl (120 mM KCl) and high KCl (250 mM KCl), at 58° C. and 62° C.

TABLE 1
Activity of engineered RTs

| | Full-Length Product (% total) | | |
|---|---|---|---|
| reverse | Standard Salt/ | High Salt/ | Standard Salt/ |
| transcriptase | 58° C. | 58° C. | 62° C. |
| CAV RT1 | 87% | 0% | 0% |
| ERT1 | 83% | 86% | 83% |
| ERT3 | 82% | 86% | 81% |
| ERT2 | 85% | 79% | 80% |
| ERT4 | 87% | 78% | 78% |

### Example 2: High Yield cDNA Synthesis Under a Variety of Reaction Buffer Conditions

The engineered Reverse Transcriptase 1 (ERT1) can efficiently synthesize full-length cDNA product within 10 minutes at 55° C. In vitro transcribed poly(A) 1 kb or 4 kb RNA templates were used to investigate full-length cDNA synthesis in a variety of buffers with pH ranging from 7.5 to 8.5, KCl concentration ranging from 40 mM to 300 mM, and MgCl₂ concentration from 1.5 to 6 mM. After first-strand cDNA synthesis, an aliquot of the cDNA products was used to make full-length ds cDNA in the presence of a 5′ specific primer. Equal volume of ds cDNA was analyzed on an agarose gel.

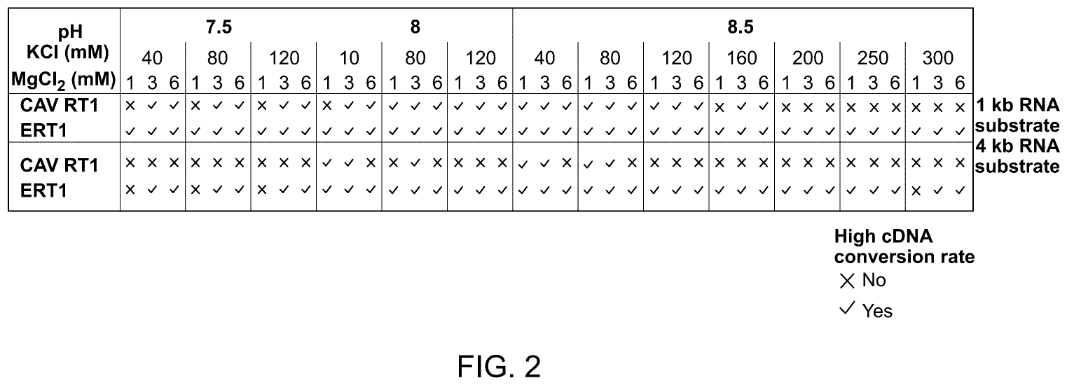

Results are shown in FIG. 2. The check (V) indicates visible ds cDNA band with a correct size on the gel and the cross (x) indicates non-visible ds cDNA band with a correct size or smear on the gel. A commercially available RT (CAV RT1) was used as a control. The engineered Reverse Transcriptase exhibits broad buffer tolerance, permitting high yield cDNA synthesis.

### Example 3: Inhibitor Tolerance of ERT

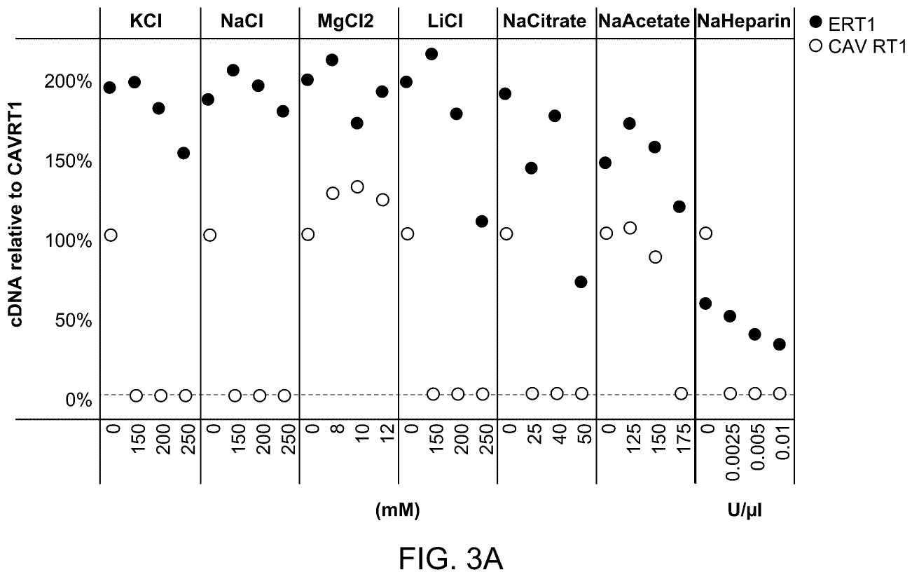

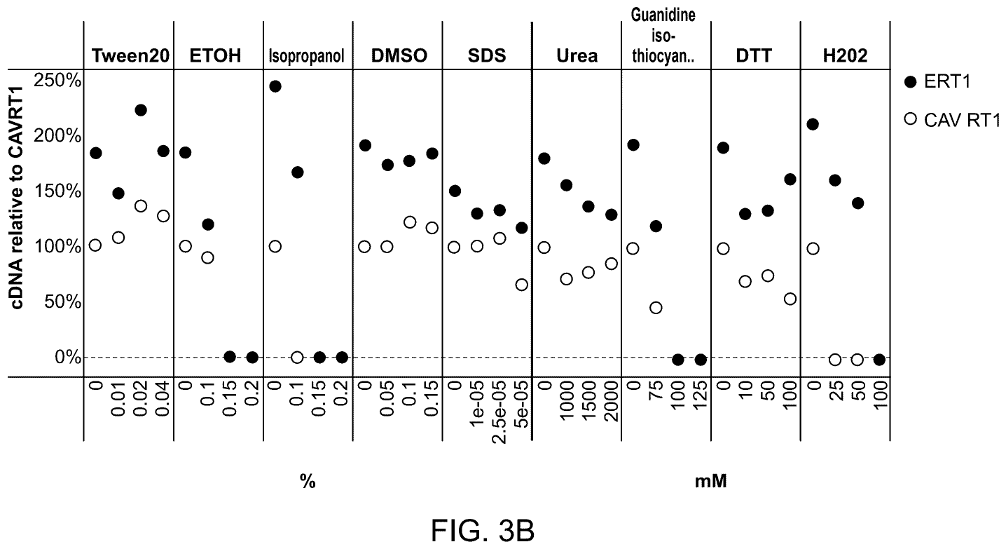

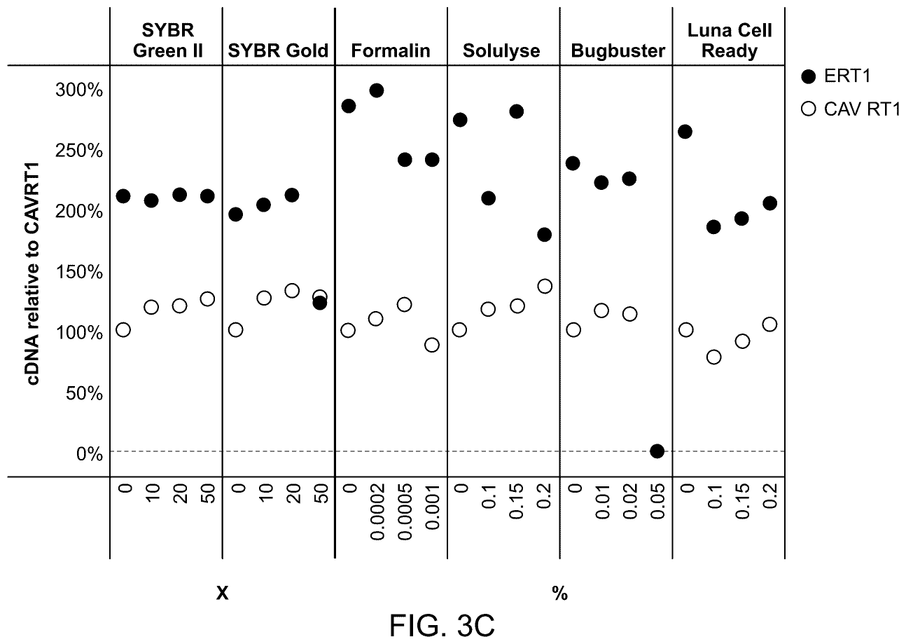

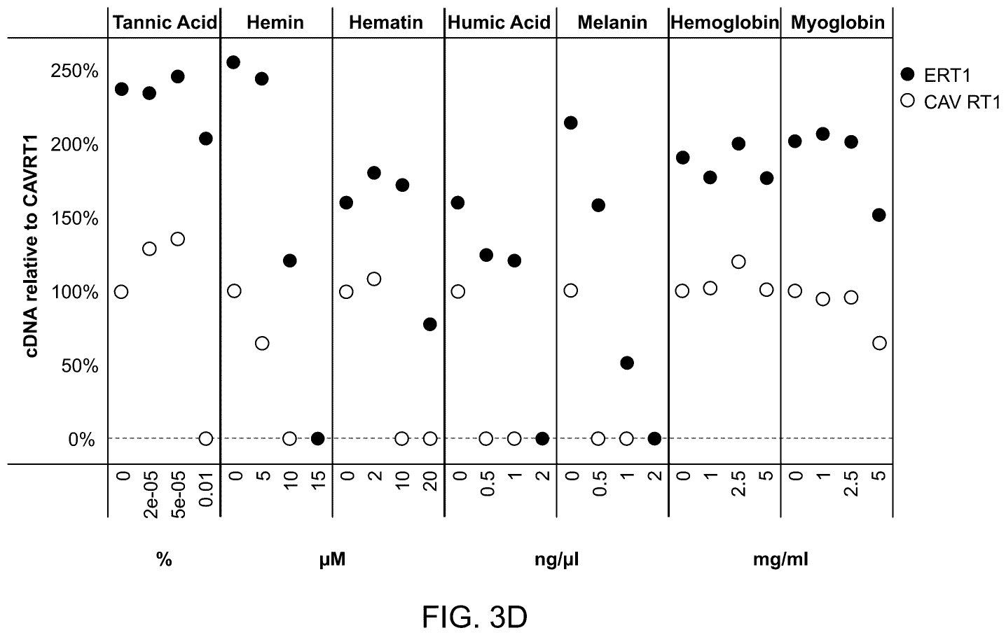

Tolerance of ERT1 to various salts that can inhibit RT reactions, including concentrations of KCl, NaCl, MgCl₂, LiCl, Na citrate, Na acetate, and Na heparin, was examined. Results are shown in FIG. 3A. In vitro transcribed poly(A) 1 kb RNA templates were used to investigate full-length cDNA synthesis in the presence of different salts at various concentrations as depicted in FIG. 3A. After first-strand cDNA synthesis, an aliquot of the cDNA products was used to make full-length ds cDNA in the presence of a 5′ specific primer. Equal volume of ds cDNA was analyzed on an agarose gel. The yield of cDNA was quantified using a GelAnalyzer instrument and normalized to the yield of cDNA synthesized with CAV RT1 in the absence of inhibitor. These results show that ERT1 can synthesize full-length cDNA product within 10 minutes at 55° C. in the presence of a variety of salts. Performance of ERT1 exceeded that of CAV RT1 as indicated by the percentage of cDNA produced relative to CAV RT1.

Tolerance of ERT1 to various substances sometimes present in samples to be treated with an RT, including Tween 20, ethanol, isopropanol, DMSO, SDS, urea, guanidine iso thiocyanate, DTT and hydrogen peroxide, was examined. Results are shown in FIG. 3B. In vitro transcribed poly(A) 1 kb RNA templates were used to investigate full-length cDNA synthesis in the presence of different inhibitors with a concentration gradient. After first-strand cDNA synthesis, an aliquot of the cDNA products was used to make full-length ds cDNA in the presence of a 5′ specific primer. Equal volume of ds cDNA was analyzed on an agarose gel. The yield of cDNA was quantified using a GelAnalyzer instrument and normalized to the yield of cDNA synthesized with CAV RT1 in the absence of inhibitor. These results show that ERT1 can synthesize full-length cDNA product within 10 minutes at 55° C. in the presence of a variety of commonly used chemical inhibitors. Performance of ERT1 exceeded that of CAV RT1 as indicated by the percentage of cDNA produced relative to CAV RT1.

Tolerance of ERT1 to various dyes and cell lysis substances, including SYBR Green II, SYBR Gold, formalin, Solulyse reagent, BugBuster reagent, and Luna Cell Ready reagent, was examined. Results are shown in FIG. 3C. In vitro transcribed poly(A) 1 kb RNA templates were used to investigate full-length cDNA synthesis in the presence of DNA/RNA binding dye and lysis reagent with a concentration gradient. After first-strand cDNA synthesis, an aliquot of the cDNA products was used to make full-length ds cDNA in the presence of a 5′ specific primer. Equal volume of ds cDNA was analyzed on an agarose gel. The yield of cDNA was quantified using a GelAnalyzer instrument and normalized to the yield of cDNA synthesized with CAV RT1 in the absence of inhibitor. These results show that ERT1 can synthesize full-length cDNA product within 10 minutes at 55° C. in the presence of DNA/RNA binding dyes and lysis reagent over a concentration gradient. Performance of ERT1 exceeded that of CAV RT1 as indicated by the percentage of cDNA produced relative to CAV RT1.

Tolerance of ERT1 to various environmental and animal sample components, including tannic acid, hemin, hematin, humic acid, melanin, hemoglobin, and myoglobin, was examined. Results are shown in FIG. 3D. In vitro transcribed poly(A) 1 kb RNA templates were used to investigate full-length cDNA synthesis. After first-strand cDNA synthesis, an aliquot of the cDNA products was used to make full-length ds cDNA in the presence of a 5′ specific primer. Equal volume of ds cDNA was analyzed on an agarose gel. The yield of cDNA was quantified using a GelAnalyzer instrument and normalized to the yield of cDNA synthesized with CAV RT1 in the absence of inhibitor. These results show that ERT1 can synthesize full-length cDNA product within 10 minutes at 55° C. in the presence of a variety of environmental and animal sample components. Performance of ERT1 exceeded that of CAV RT1 as indicated by the percentage of cDNA produced relative to CAV RT1.

### Example 4. Long cDNA Synthesis Using ERT1

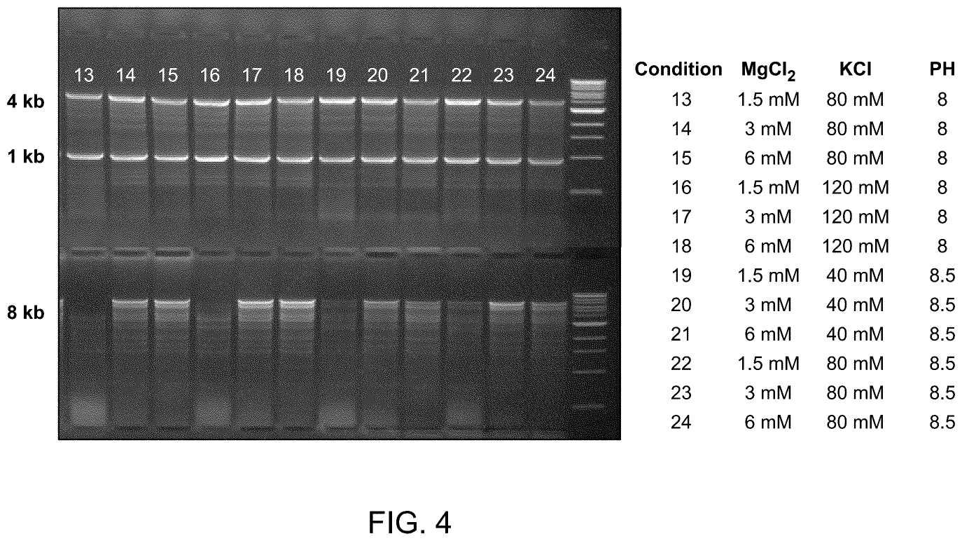

ERT1 can efficiently synthesize 1 kb, 4 kb as well as 8 kb full-length cDNA product within 10 minutes at 55° C. In vitro transcribed poly(A) 1 kb, 4 kb or 8 kb RNA templates were used to investigate full-length cDNA synthesis in a variety of buffers with pH ranging from 8 to 8.5, KCl concentration ranging from 40 mM to 120 mM, and MgCl₂ concentration from 1.5 mM to 6 mM (see FIG. 4). After first-strand cDNA synthesis, an aliquot of the cDNA products was used to make full-length ds cDNA in the presence of a 5′ specific primer. Equal volume of ds cDNA was analyzed on an agarose gel. Under all the buffer conditions, 1 kb and 4 kb full length cDNA can be efficiently synthesized, while full length and partial cDNA products can be synthesized under selected buffer condition as shown in FIG. 4. A general-purpose reaction mixture suitable for generating long cDNA (e.g., about 8 kb) could therefore contain about 50 mM Tris-HCl, about 3 mM MgCl₂, about 0.02% Tween-20, about 120 mM KCl, at about pH 8.5. The engineered Reverse Transcriptase ERT1 exhibits broad buffer tolerance, permitting high yield cDNA synthesis for long cDNA.

### Example 5. Evaluation of an RT in One-Step RT-qPCR

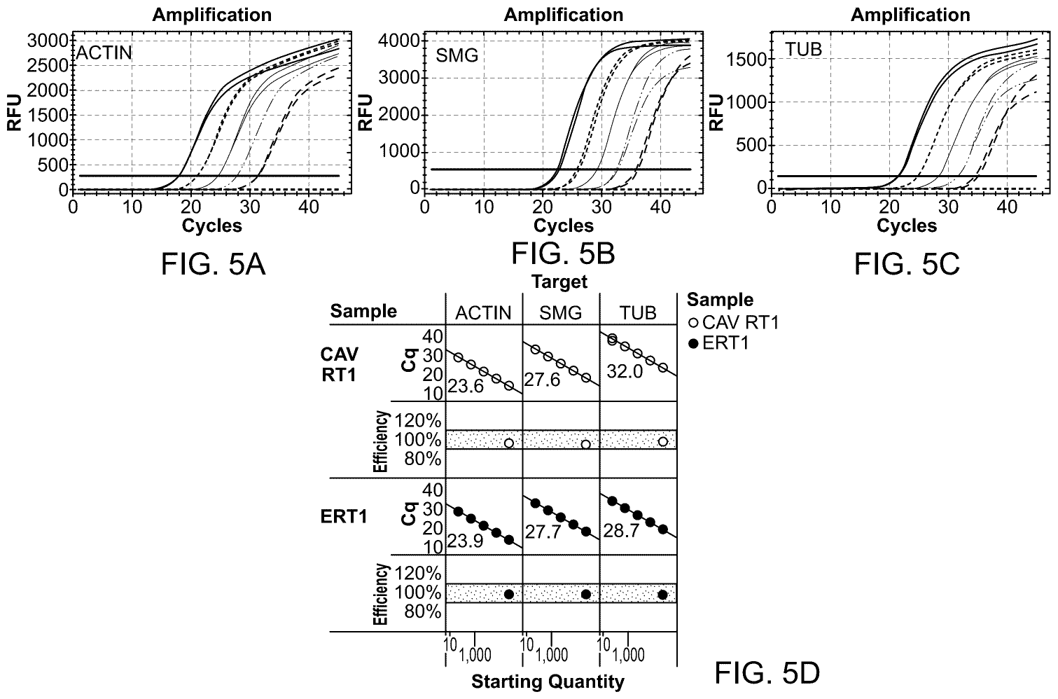

ERT1 was used in a One-Step RT-qPCR reaction with probe-based detection. Results are shown in FIG. 5A-5D. Typical amplification curves for the amplicons Actin, SMG and TUB are shown in FIGS. 5A, 5B, and 5C, respectively. Different lines indicate different amounts of cDNA used in 20 μl qPCR reaction. The RNA input in the 20 μl cDNA synthesis reaction was (from left to right): 1 μg RNA, 100 ng RNA, 10 ng RNA, 1 ng RNA and 100 pg RNA). Assuming 100% cDNA conversion rate, (from left to right): 50 ng cDNA, 5 ng cDNA, 500 pg cDNA, 50 pg cDNA and 5 pg cDNA used in 20 μl qPCR reaction. As shown in FIG. 5D, Cqs were plotted against (linearity and efficiency) the RNA input across 5-log (50 ng to 5 pg Jurkat RNA inputs). Cq average and efficiency for each target were calculated through the 5-log (FIG. 5D). CAV RT1 was used as a comparison with a comparable Cq for Actin and SMG and earlier Cq for TUB. The engineered Reverse Transcriptase provides sensitive and accurate detection and quantitation across a wide range of RNA inputs in One-Step RT-qPCR.

### Example 6. Evaluation of an RT in Two-Step RT-PCR

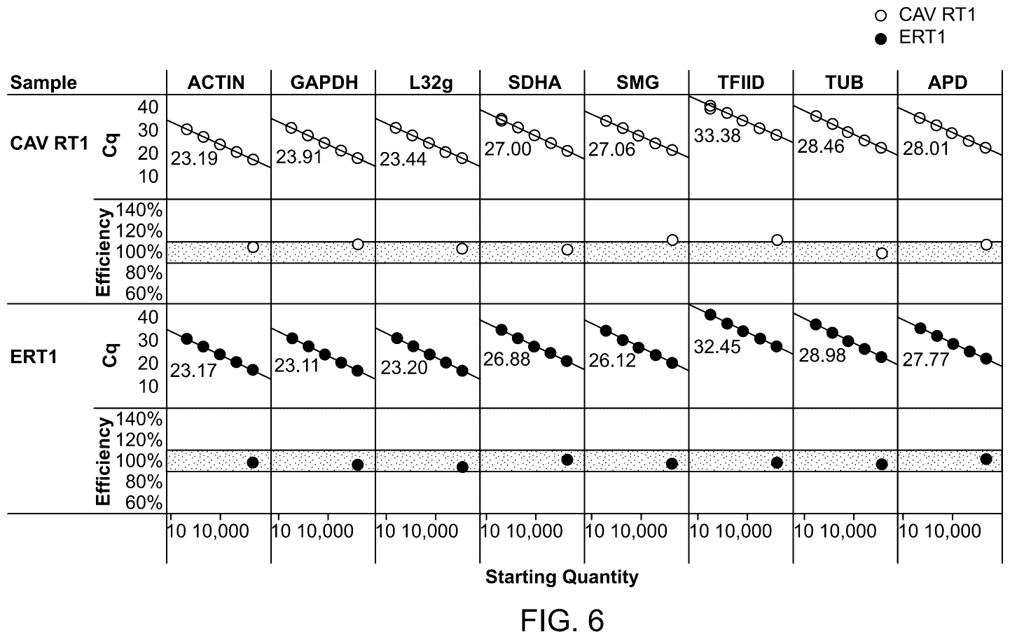

ERT1 was used in a Two-Step RT-qPCR reaction (25° C./2 minutes, 55° C./10 minutes, 95° C./1 minutes), followed by probe-based detection. Results are shown in FIG. 6. cDNA synthesis was performed with the RNA input across 5-log (1 μg to 100 pg Hela RNA). 1 μl cDNA was then quantitated by qPCR using Luna Universal Probe qPCR Master Mix (M3004, New England Biolabs, Inc.). Cqs were plotted against the RNA input across 5-log. Cq average and efficiency for each target were also calculated through the 5-log and shown in FIG. 6. CAV RT1 was used as a comparison, with a comparable Cq for most targets and earlier Cq for SMG and TFIID. The engineered Reverse Transcriptase provides sensitive and accurate detection and quantitation across a wide range of RNA input in Two-Step RT-qPCR.

### Example 7. ERT Faster Performance

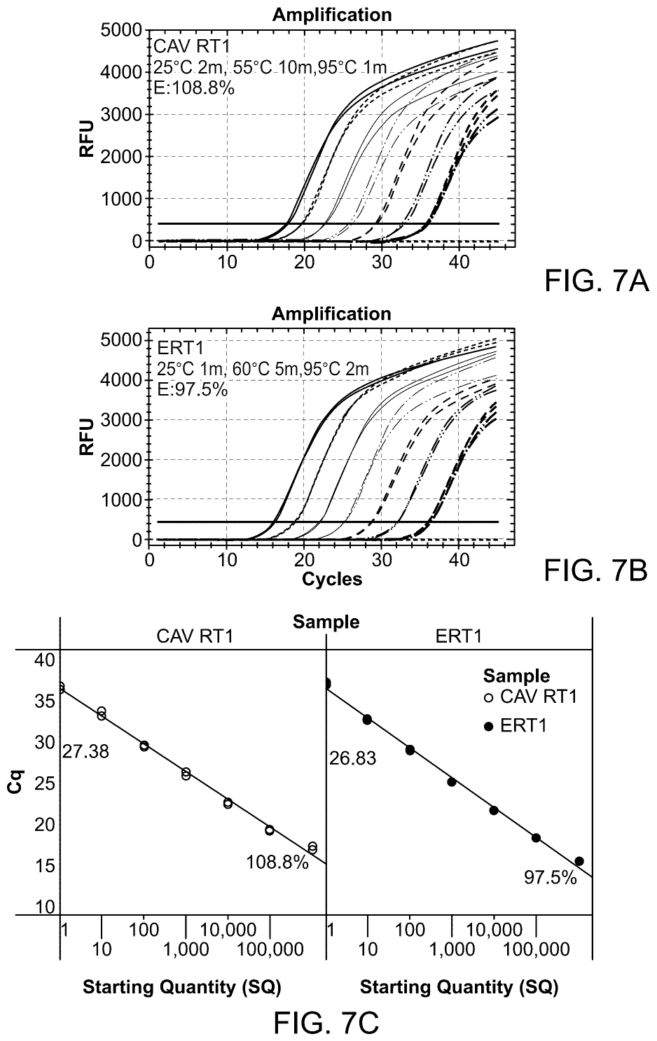

ERT1 was used in an 8-minute Two-Step RT-qPCR reaction (25° C./1 minute, 60° C./5 minutes, 95° C./2 minutes), followed by probe-based detection. Results are shown in FIG. 7A-7C. cDNA synthesis was performed with the RNA input across 7-log (1 μg to 1 pg). 1 μl cDNA was then quantitated by qPCR using Luna Universal Probe qPCR Master Mix (M3004, New England Biolabs, Inc.). Typical amplification curves for the amplicon Actin are shown in FIG. 7A (control CAV RT1) and FIG. 7B (ERT1). Different lines indicate different amounts of cDNA used in 20 μl qPCR reaction. The RNA input in the 20 μl cDNA synthesis reaction was (from left to right): 1 μg RNA, 100 ng RNA, 10 ng RNA, 1 ng RNA, 100 pg RNA, 10 pg RNA, 1 pg RNA). Assuming 100% cDNA conversion rate, (from left to right): 50 ng cDNA, 5 ng cDNA, 500 pg cDNA, 50 pg cDNA, 5 pg cDNA, 0.5 pg cDNA and 0.05 pg cDNA used in 20 μl qPCR reaction. Cqs were plotted against the RNA input across 7-log. Cq average and efficiency were also calculated through the 7-log and shown in FIG. 7C. CAV RT1 was used as a comparison using an established 13 min fast cycling protocol (25° C./2 minutes, 55° C./10 minutes, 95° C./1 minute). The engineered Reverse Transcriptase provides sensitive and accurate detection and quantitation across a wide range of RNA input in Two-Step RT-qPCR with an 8 minute cDNA synthesis protocol at an elevated temperature, with improved performance relative to CAV RT1, which has a 13 minute protocol.

### Example 8. Five-Plex Two-Plex RT-qPCR

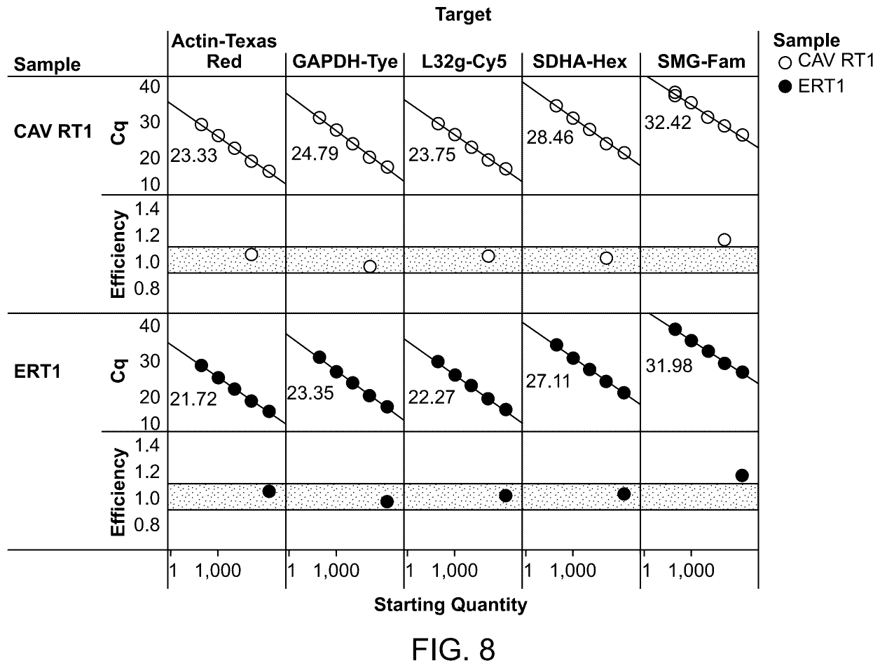

ERT1 was used in an 8-minute Two-Step RT-qPCR reaction (25° C./1 minute, 60° C./5 minutes, 95° C./2 minutes), followed by probe-based 5-plex detection. cDNA synthesis was performed with the RNA input across 5-log (1 μg to 100 pg). The samples were actin, glyceraldehyde-3-phosphate dehydrogenase (GAPDH), ribosomal protein L32 (L32g), succinate dehydrogenase complex, subunit A (SDHA), SMG1 nonsense mediated mRNA decay associated PI3K related kinase (SMG). Amplicons were relatively small, with the longest being GAPDH at 226 bp. 1 μl cDNA from each reaction was then quantitated by qPCR using Luna Universal Probe qPCR Master Mix (M3004, New England Biolabs, Inc.). Cqs were plotted against the RNA input across 5-log. Cq average and efficiency were also calculated through the 5-log and shown in FIG. 8. CAV RT1 was used as a comparison with an established 13 minute fast cycling protocol. ERT1 showed earlier Cq for all five targets in the multiplex assay. Lower Cq suggests higher cDNA conversion efficiency with the same RNA inputs.

### Example 9. Even Coverage of a 16 kb RNA Transcript

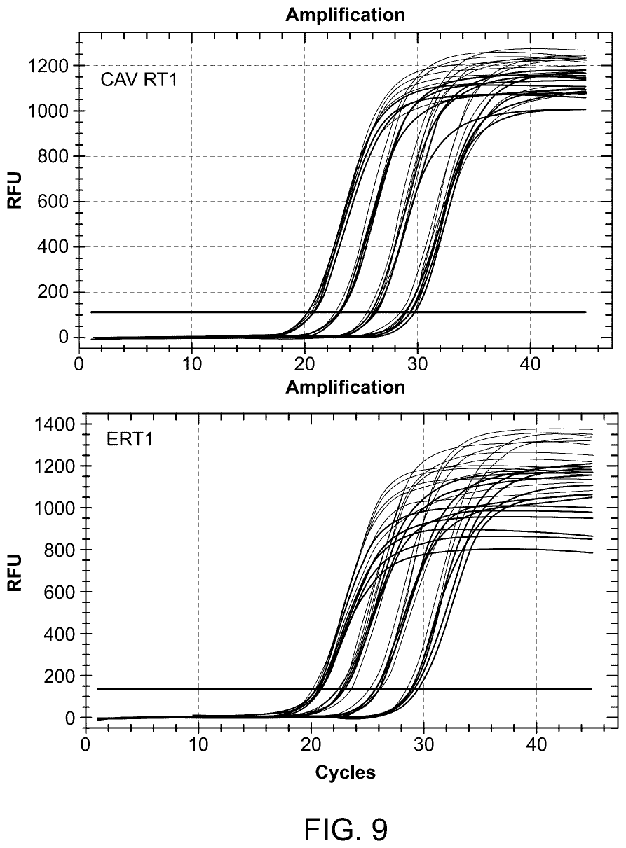

ERT1 was used in an 8-minute Two-Step RT-qPCR reaction (25° C./1 minute, 60° C./5 minutes, 95° C./2 minutes), followed by dye-based detection. cDNA synthesis was performed with the RNA input across 4-log (1 μg to 1 ng). 1 μl cDNA was then quantitated by qPCR using Luna Universal qPCR Master Mix (M3003, New England Biolabs, Inc.). Typical amplification curves for the amplicon HerC are shown in FIG. 9. 6 sets of qPCR primer sets were designed to evenly cover the full 16 kb RNA transcript. CAV RT1 was used as a comparison with the established 13 minute fast cycling protocol. As depicted in FIG. 9, ERT1 showed almost identical even coverage for all the six sets of primers in the evaluation as CAV RT1. Different lines indicate different amounts of cDNA used in 20 μl qPCR reaction. The RNA input in the 20 μl cDNA synthesis reaction was (from left to right): 1 μg RNA, 100 ng RNA, 10 ng RNA, and 1 ng RNA). Assuming 100% cDNA conversion rate, (from left to right): 50 ng cDNA, 5 ng cDNA, 500 pg cDNA and 50 pg cDNA used in 20 μl qPCR reaction. The engineered Reverse Transcriptase provides even coverage detection and quantitation across a wide range of RNA input in Two-Step RT-qPCR with an 8 minute cDNA synthesis protocol at an elevated temperature.

### Example 10. Template Switching

Template switching reactions were performed using components from the NEBNext® Single Cell/Low Input cDNA Synthesis & Amplification Module (E6421, New England Biolabs). In this protocol, reverse transcription is performed using a reverse transcription primer containing a 3′ poly(T) sequence and a 5′ tail sequence that allows for annealing of a PCR primer. A template switching oligo (TSO) is used to incorporate a known sequence to the 3′ end of the cDNA for PCR priming when template switching is performed. Amplification of full-length cDNA products is only observed when the sequence of the TSO is incorporated into the cDNA strand.

Reactions were performed using 4 ng of Universal Human Reference RNA (740000, Agilent) containing SIRV-Set 4 spike-in RNAs (141, Lexogen), enriched for poly(A)-containing RNA using the NEBNext® High Input Poly(A) mRNA Isolation Module (E3370, New England Biolabs). Reverse transcription reactions were performed in 1× Template Switching RT Buffer (B0466, New England Biolabs) containing Murine RNase Inhibitor (M0314, New England Biolabs), T4 Gene 32 Protein (M0300, New England Biolabs), NEBNext® Single Cell RT Primer Mix (E6422, New England Biolabs, 5′-AAGCAGTGGTATCAACGCAGAGTACTTTTTTTTTTTV-3′) (SEQ ID NO:5), NEBNext® Template Switching Oligo (E6424, New England Biolabs, 5′-GCTAATCATTGCAAGCAGTGGTATCAACGCAGAGTACATrGrGrG-3′) (SEQ ID NO:6), and reverse transcriptase. Template Switching RT Enzyme Mix (M0466, New England Biolabs) served as a positive control for template switching activity (TS Control). Reverse transcription reactions were performed for 90 minutes at the indicated temperature, followed by heat denaturation at 85° C. for 5 minutes. Amplification of cDNA was performed for 10 cycles following manufacturer protocol using primers with the sequence 5′-AAGCAGTGGTATCAACGCAGAGT-3′ (SEQ ID NO:7). cDNA products were analyzed using Agilent® High Sensitivity D5000 ScreenTape® System (5067, Agilent) on an Agilent 4200 TapeStation® (G2991, Agilent).

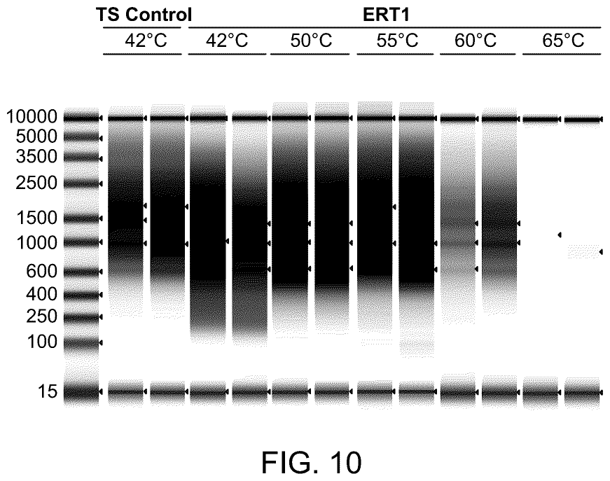

As shown in FIG. 10, reactions performed with ERT1 produce robust amplification of cDNA compared to the control, even at higher reaction temperatures, indicating strong template switching activity of ERT1. The full-length cDNA products were then sequenced using short-read and long-read sequencing.

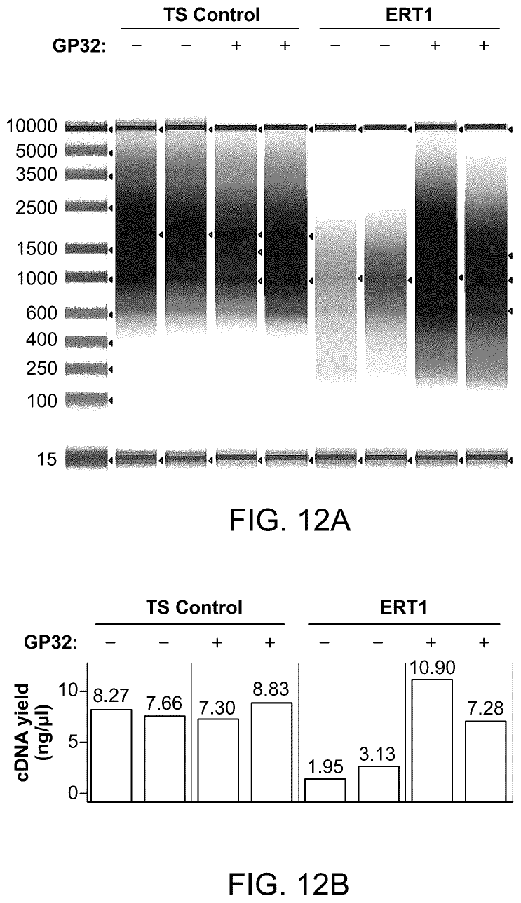

Experiments were performed to determine whether template switching activity of ERT1 is altered by the presence of T4 Gene 32 Protein (GP32). As is depicted in FIGS. 12A and 12B, the template switching reaction with ERT1 produces a higher cDNA yield in the presence of GP32 (+ lanes) while this is not observed for the control commercially available reverse transcriptase enzyme.

Sequencing libraries were prepared from cDNA using NEBNext Ultra II FS DNA Library Prep Kit for Illumina (E7805, New England Biolabs) and sequenced on an Illumina NextSeq® 500 (paired-end, 75 bases). Reads were aligned to the hg38 reference genome using RNA STAR and transcript coverage was calculated from the 1,000 most-abundant transcripts using the CollectRnaSeqMetrics (Picard) tool. Shown are the average of two replicates.

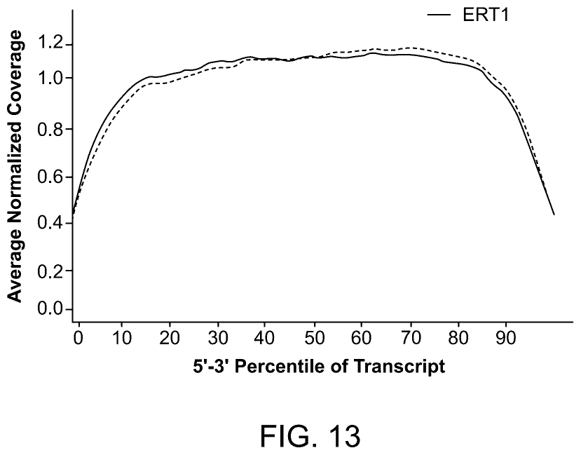

FIG. 13 shows that transcript coverage of cDNA produced from template switching reactions with template switching (TS) control enzyme or ERT1 as determined from aligned sequencing reads. ERT1 produces more even transcript coverage compared to the control enzyme, with improved representation of the 5′ RNA sequence, indicating improved reverse transcription activity.

### Example 11. Improved Thermal Stability of ERT1

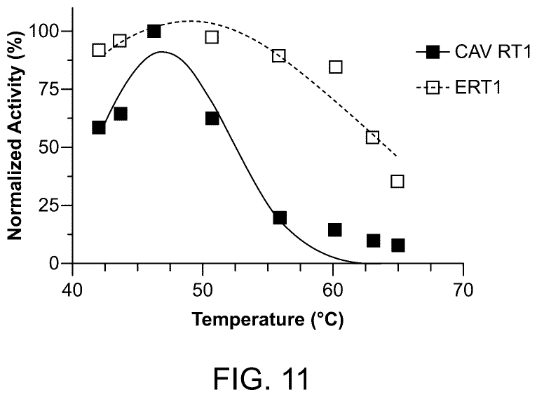

Temperature dependence of ERT1 and commercially available RT (CAV RT1) was determined by measuring full-length cDNA product formation. Synthesis reactions were assembled with 300 nM 1 kb RNA, 450 nM of DNA primer, 500 UM dNTPs, 1 U/μl RNase Inhibitor (NEB M0314) and 1× optimal buffer. Reactions were initiated with 37.5 nM of Enzyme and incubated for 30 minutes at 42° C., 43.6° C., 46.3° C., 50.7° C., 55.9° C., 60.2° C., 63.1° C., or 65° C. Full-length cDNA products were measured with 450 nM of a FAM-IABκFQ molecular beacon that emits fluorescence (excitation: 480/20 nm, emission: 540/20 nm) upon annealing to the 3′ terminus of the full-length cDNA product. Activity was calculated from duplicate readings and normalized according to the maximum fluorescence reading of each enzyme. As shown in FIG. 11, the synthetic ERT1 produced more full-length cDNA products at elevated temperature compared to the CAV RT1.

While the present invention has been described with reference to the specific embodiments thereof, it should be understood by those skilled in the art that various changes may be made, and equivalents may be substituted without departing from the true spirit and scope of the invention. In addition, many modifications may be made to adapt a particular situation, material, composition of matter, process, process step or steps, to the objective, spirit, and scope of the present invention. All such modifications are intended to be within the scope of the claims appended hereto.

SEQUENCES

SEQ ID NO: 1
AWLQDFPQAWAETGGMGLAKCQAPVIIELKPTATPVSIRQYPMSKQARQ
GIRPHIQRLLEQGILVPCQSPWNTPLLPVKKPGTNDYRPVQDLREVNKR
VEDIHPTVPNPYNLLSTLPPDRTWYTVLDLKDAFFCLPLAPESQPLFAF
EWRDPERGISGQLTWTRLPQGFKNSPTLFQEALHRDLADFRVQHPEVTL
LQYVDDLLLAAPTEEACLQGTRALLQELGDLGYRASAKKAQICQKEVTY
LGYILSEGQRWLTEARKETVMQIPTPKTPRQVRQFLGTAGFCRLWIPGF
AELAAPLYALTKEGTPFTWGPEHQKAFEALKKALLSAPALGLPDLTKPF
TLFVDEKQGIAKGVLTQKLGPQKRPVAYLSKKLDPVAAGWPPCLRAIAA
VAILVKDAGKLTLGQPLTVITPHALEAIVRQPPDRWLTNARMTHYQALL
LDTDRVQFGPPVTLNPATLLPVPSDEPPAHDCLEILAETHGTRPDLTDQ
PLPDADLTWYTGGSSFIQEGKRRAGAAVVDGHRVIWAQSLPPGTSAQKA
QLIALTKALELSEGKKVNIYTNSRYAFATAHVHGAIYRRRGLLTSEGKE
IKNKAEILALLEALFLPKRVAIIHCPGHQKGNSPVAKGNRQADQVARQA
AMGTTTTLT
SEQ ID NO: 2
SWLQQFPQVWAEQAGMGLAKQVPPVVVELKADATPVSVRQYPMSKQARE
GIRPHIQRLLDQGILVPCQSPWNTPLLPVKKPGTNDYRPVQDLREVNKR
VQDIHPTVPNPYNLLSSLPPSRTWYTVLDLKDAFFCLPLHPNSQPLFAF
EWRDPESGQTGQLTWTRLPQGFKNSPTLFQEALHRDLAPFRAQNPQVTL
LQYVDDLLVAAATKEDCQQGTQRLLQELSDLGYRVSAKKAQICQREVTY
LGYTLRGGKRWLTEARKKTVMQIPTPTTPRQVRQFLGTAGFCRLWIPGF
ATLAAPLYPLTKEKVPFTWTEEHQRAFEAIKKALLSAPALALPDLTKPF
TLYVDERAGVARGVLTQTLGPQKRPVAYLSKKLDPVASGWPTCLKAIAA
VALLIKDADKLTMGQNVTVIAPHALESIVRQPPDRWMTNARMTHYQSLL
LNERVSFAPPAILNPATLLPVESDETPVHQCSEILAEETGTRPDLTDQP
WPGAPTWYTGGSSFIVEGKRKAGAAVVDGKRVIWASSLPEGTSAQKAQL
IALTQALRLAEGKSINIYTNSRYAFATAHVHGAIYRQRGLLTSAGKDIK
NKEEILALLEAIHLPKRVAIIHCPGHQRGTSPVAKGNRMADQVAKQAAQ
GTMILT
SEQ ID NO: 3
AWLQDFPQAWAETGGMGLAKCQAPVIIELKPTATPVSIRQYPMSKQARQ
GIRPHIQRLLEQGILVPCQSPWNTPLLPVKKPGTNDYRPVQDLREVNKR
VEDIHPTVPNPYNLLSTLPPDRTWYTVLDLKDAFFCLPLAPESQPLFAF
EWRDPERGISGQLTWTRLPQGFKNSPTLFQEALHRDLADFRVRHPEVTL
LQYVDDLLLAAPTEEACLQGTRALLQELGDLGYRASAKKAQICQKEVTY
LGYILSEGQRWLTEARKETVMQIPTPKTPRQVRQFLGTAGFCRLWIPGF
AELAAPLYALTKEGTPFTWGPEHQKAFEALKKALLSAPALGLPDLTKPF
TLFVDEKQGIAKGVLTQKLGPQKRPVAYLSKKLDPVAAGWPPCLRAIAA
VAILVKDAGKLTLGQPLTVITPHALEAIVRQPPDRWLTNARMTHYQALL
LDTRRVQFGPPVTLNPATLLPVPSDEPPAHDCLEILAETHGTRPDLTDQ
PLPDADLTWYTGGSSFIQKGKRRAGAAVVDGHRVIWAQSLPPGTSAQKA
QLIALTKALELSEGKKVNIYTNSRYAFATAHVHGAIYRRRGLLTSEGKK
IKNKAEILALLEALFLPKRVAIIHCPGHQKGNSPVAKGNRQADQVARQA
AMGTTTTLT
SEQ ID NO: 4
SWLQQFPQVWAEQAGMGLAKQVPPVVVELKADATPVSVRQYPMSKQARE
GIRPHIQRLLDQGILVPCQSPWNTPLLPVKKPGTNDYRPVQDLREVNKR
VQDIHPTVPNPYNLLSSLPPSRTWYTVLDLKDAFFCLPLHPNSQPLFAF
EWRDPESGQTGQLTWTRLPQGFKNSPTLFQEALHRDLAPFRARNPQVTL
LQYVDDLLVAAATKEDCQQGTQRLLQELSDLGYRVSAKKAQICQREVTY
LGYTLRGGKRWLTEARKKTVMQIPTPTTPRQVRQFLGTAGFCRLWIPGF
ATLAAPLYPLTKEKVPFTWTEEHQRAFEAIKKALLSAPALALPDLTKPF
TLYVDERAGVARGVLTQTLGPQKRPVAYLSKKLDPVASGWPTCLKAIAA
VALLIKDADKLTMGQNVTVIAPHALESIVRQPPDRWMTNARMTHYQSLL
LNRRVSFAPPAILNPATLLPVESDETPVHQCSEILAEETGTRPDLTDQP
WPGAPTWYTGGSSFIVKGKRKAGAAVVDGKRVIWASSLPEGTSAQKAQL
IALTQALRLAEGKSINIYTNSRYAFATAHVHGAIYRQRGLLTSAGKKIK
NKEEILALLEAIHLPKRVAIIHCPGHQRGTSPVAKGNRMADQVAKQAAQ
GTMILT
SEQ ID NO: 5
AAGCAGTGGTATCAACGCAGAGTACTTTTTTTTTTTTTTTTTTTTTTTT
TTTTTTV
SEQ ID NO: 6
GCTAATCATTGCAAGCAGTGGTATCAACGCAGAGTACATrGrGrG
SEQ ID NO: 7
AAGCAGTGGTATCAACGCAGAGT
SEQ ID NO: 8
MGKIEESKHHHHHHGS

What is claimed is:

1. A reverse transcriptase comprising:
an amino acid sequence having at least 90% identity with SEQ ID NO:1; or
an amino acid sequence having at least 90% identity with SEQ ID NO:2.

2. The reverse transcriptase of claim 1, wherein the amino acid sequence has at least 95% sequence identity with SEQ ID NO:1.

3. The reverse transcriptase of claim 1, wherein the amino acid sequence has at least 95% sequence identity with SEQ ID NO:2.

4. The reverse transcriptase of claim 1, comprising:
an amino acid sequence having at least 90% identity with SEQ ID NO:1, wherein the amino acid sequence has one or more of the following amino acid substitutions:
an R at position 190,
an R at position 445,
a K at position 509, and
a K at position 588,
wherein the positions correspond to positions in SEQ ID NO:1.

5. The reverse transcriptase of claim 4, wherein the amino acid sequence contains:
an R at position 190,
an R at position 445,
a K at position 509, and
a K at position 588,
wherein the positions correspond to positions in SEQ ID NO:1.

6. The reverse transcriptase of claim 1, comprising:
an amino acid sequence having at least 90% identity with SEQ ID NO:2, wherein the amino acid sequence has one or more of following amino acid substitutions:
an R at position 190,
an R at position 444,
a K at position 507, and
a K at position 586,
wherein the positions correspond to positions in SEQ ID NO:2.

7. The reverse transcriptase of claim 6, wherein the amino acid sequence contains
an R at position 190,
an R at position 444,
a K at position 507, and
a K at position 586,
wherein the positions correspond to positions in SEQ ID NO:2.

8. The reverse transcriptase of claim 1, wherein the reverse transcriptase is a fusion protein comprising: (i) a polymerase domain comprising the amino acid sequence; and (ii) an exogenous amino acid sequence.

9. A reverse transcriptase fusion protein comprising:
(i) a polymerase domain having reverse transcriptase activity, comprising an amino acid sequence that is a portion of:
an amino acid sequence having at least 90% identity with SEQ ID NO:1; and
an amino acid sequence having at least 90% identity with SEQ ID NO:2; and
(ii) an exogenous amino acid sequence.

10. The reverse transcriptase of claim 8, wherein the exogenous amino acid sequence comprises a purification tag.

11. A composition comprising a reverse transcriptase of claim 1.

12. The composition of claim 11, further comprising one or more of: dNTPs, a buffering agent, an oligonucleotide, an aptamer, a template switching oligonucleotide, a DNA polymerase, an RNA template and a DNA template.

13. The composition of claim 11, wherein reverse transcriptase is associated with a solid support.

14. The composition of claim 11, wherein the composition is in a form selected from lyophilized, dried, and in solution.

15. A kit comprising:
(i) a reverse transcriptase of claim 1; and
(ii) a component selected from a buffering agent, a cationic salt, dNTPs, a primer, an aptamer, a detergent, a template switching oligonucleotide, and nucleic acid binding protein.

16. The kit of claim 15, wherein the reverse transcriptase is in a form selected from lyophilized, dried, and in solution.

17. The kit of claim 15, wherein one or more components is associated with a solid support.

18. A kit comprising:
(i) a reverse transcriptase fusion protein of claim 9; and
(ii) a component selected from a buffering agent, a cationic salt, dNTPs, a primer, an aptamer, a detergent, a template switching oligonucleotide, and nucleic acid binding protein.

19. A method comprising:
incubating a reaction mixture comprising
(i) a reverse transcriptase of claim 1,
(ii) a primer,
(ii) a target nucleic acid, and
(iii) dNTPs,
under conditions suitable for the reverse transcriptase to copy the target nucleic acid to generate copied DNA.

20. The method of claim 19, wherein the incubating is done at a temperature of between 55° C.-65° C.

21. The method of claim 19, wherein the primer is selected from an oligo(dT) primer, a random primer, a target-specific primer.

22. The method of claim 19, wherein the target nucleic acid is RNA.

23. The method of claim 19, wherein the target nucleic acid is DNA.

24. The method of claim 18, wherein the target nucleic acid is an RNA, and further comprising incubating the generated copied DNA with an enzyme to generate second copied DNA.

25. The method of claim 19, further comprising quantifying the amount of the copies of the target nucleic acid.

26. The method of claim 22, wherein the RNA template is of a size of at least about 1 kb, and full length copied DNA is generated within a time period selected from 10 minutes, 9 minutes, 8 minutes, less than 10 minutes, less than 9 minutes, and less than 8 minutes.

27. A method comprising:
incubating a reaction mixture comprising
(i) a reverse transcriptase fusion protein of claim 9,
(ii) a primer,
(ii) a target nucleic acid, and
(iii) dNTPs,
under conditions suitable for the reverse transcriptase to copy the target nucleic acid to generate copied DNA.


SEQUENCE LISTING (8 sequences)

SEQ ID NO: 1 - protein, 646 aa, synthetic construct
AWLQDFPQAWAETGGMGLAKCQAPVIIELKPTATPVSIRQYPMSKQARQGIRPHIQRLLEQGILVPCQSPWNTPLLPVKKPGTNDYRPVQDLREVNKRVEDIHPTVPNPYNLLSTLPPDRTWYTVLDLKDAFFCLPLAPESQPLFAFEWRDPERGISGQLTWTRLPQGFKNSPTLFQEALHRDLADFRVQHPEVTLLQYVDDLLLAAPTEEACLQGTRALLQELGDLGYRASAKKAQICQKEVTYLGYILSEGQRWLTEARKETVMQIPTPKTPRQVRQFLGTAGFCRLWIPGFAELAAPLYALTKEGTPFTWGPEHQKAFEALKKALLSAPALGLPDLTKPFTLFVDEKQGIAKGVLTQKLGPQKRPVAYLSKKLDPVAAGWPPCLRAIAAVAILVKDAGKLTLGQPLTVITPHALEAIVRQPPDRWLTNARMTHYQALLLDTDRVQFGPPVTLNPATLLPVPSDEPPAHDCLEILAETHGTRPDLTDQPLPDADLTWYTGGSSFIQEGKRRAGAAVVDGHRVIWAQSLPPGTSAQKAQLIALTKALELSEGKKVNIYTNSRYAFATAHVHGAIYRRRGLLTSEGKEIKNKAEILALLEALFLPKRVAIIHCPGHQKGNSPVAKGNRQADQVARQAAMGTTTTLT

SEQ ID NO: 2 - protein, 643 aa, synthetic construct
SWLQQFPQVWAEQAGMGLAKQVPPVVVELKADATPVSVRQYPMSKQAREGIRPHIQRLLDQGILVPCQSPWNTPLLPVKKPGTNDYRPVQDLREVNKRVQDIHPTVPNPYNLLSSLPPSRTWYTVLDLKDAFFCLPLHPNSQPLFAFEWRDPESGQTGQLTWTRLPQGFKNSPTLFQEALHRDLAPFRAQNPQVTLLQYVDDLLVAAATKEDCQQGTQRLLQELSDLGYRVSAKKAQICQREVTYLGYTLRGGKRWLTEARKKTVMQIPTPTTPRQVRQFLGTAGFCRLWIPGFATLAAPLYPLTKEKVPFTWTEEHQRAFEAIKKALLSAPALALPDLTKPFTLYVDERAGVARGVLTQTLGPQKRPVAYLSKKLDPVASGWPTCLKAIAAVALLIKDADKLTMGQNVTVIAPHALESIVRQPPDRWMTNARMTHYQSLLLNERVSFAPPAILNPATLLPVESDETPVHQCSEILAEETGTRPDLTDQPWPGAPTWYTGGSSFIVEGKRKAGAAVVDGKRVIWASSLPEGTSAQKAQLIALTQALRLAEGKSINIYTNSRYAFATAHVHGAIYRQRGLLTSAGKDIKNKEEILALLEAIHLPKRVAIIHCPGHQRGTSPVAKGNRMADQVAKQAAQGTMILT

SEQ ID NO: 3 - protein, 646 aa, synthetic construct
AWLQDFPQAWAETGGMGLAKCQAPVIIELKPTATPVSIRQYPMSKQARQGIRPHIQRLLEQGILVPCQSPWNTPLLPVKKPGTNDYRPVQDLREVNKRVEDIHPTVPNPYNLLSTLPPDRTWYTVLDLKDAFFCLPLAPESQPLFAFEWRDPERGISGQLTWTRLPQGFKNSPTLFQEALHRDLADFRVRHPEVTLLQYVDDLLLAAPTEEACLQGTRALLQELGDLGYRASAKKAQICQKEVTYLGYILSEGQRWLTEARKETVMQIPTPKTPRQVRQFLGTAGFCRLWIPGFAELAAPLYALTKEGTPFTWGPEHQKAFEALKKALLSAPALGLPDLTKPFTLFVDEKQGIAKGVLTQKLGPQKRPVAYLSKKLDPVAAGWPPCLRAIAAVAILVKDAGKLTLGQPLTVITPHALEAIVRQPPDRWLTNARMTHYQALLLDTRRVQFGPPVTLNPATLLPVPSDEPPAHDCLEILAETHGTRPDLTDQPLPDADLTWYTGGSSFIQKGKRRAGAAVVDGHRVIWAQSLPPGTSAQKAQLIALTKALELSEGKKVNIYTNSRYAFATAHVHGAIYRRRGLLTSEGKKIKNKAEILALLEALFLPKRVAIIHCPGHQKGNSPVAKGNRQADQVARQAAMGTTTTLT

SEQ ID NO: 4 - protein, 643 aa, synthetic construct
SWLQQFPQVWAEQAGMGLAKQVPPVVVELKADATPVSVRQYPMSKQAREGIRPHIQRLLDQGILVPCQSPWNTPLLPVKKPGTNDYRPVQDLREVNKRVQDIHPTVPNPYNLLSSLPPSRTWYTVLDLKDAFFCLPLHPNSQPLFAFEWRDPESGQTGQLTWTRLPQGFKNSPTLFQEALHRDLAPFRARNPQVTLLQYVDDLLVAAATKEDCQQGTQRLLQELSDLGYRVSAKKAQICQREVTYLGYTLRGGKRWLTEARKKTVMQIPTPTTPRQVRQFLGTAGFCRLWIPGFATLAAPLYPLTKEKVPFTWTEEHQRAFEAIKKALLSAPALALPDLTKPFTLYVDERAGVARGVLTQTLGPQKRPVAYLSKKLDPVASGWPTCLKAIAAVALLIKDADKLTMGQNVTVIAPHALESIVRQPPDRWMTNARMTHYQSLLLNRRVSFAPPAILNPATLLPVESDETPVHQCSEILAEETGTRPDLTDQPWPGAPTWYTGGSSFIVKGKRKAGAAVVDGKRVIWASSLPEGTSAQKAQLIALTQALRLAEGKSINIYTNSRYAFATAHVHGAIYRQRGLLTSAGKKIKNKEEILALLEAIHLPKRVAIIHCPGHQRGTSPVAKGNRMADQVAKQAAQGTMILT

SEQ ID NO: 5 - DNA, 56 bp, synthetic construct
  source 1..56: mol_type = other DNA
aagcagtggtatcaacgcagagtacttttttttttttttttttttttttttttttv

SEQ ID NO: 6 - DNA, 45 bp, synthetic construct
  source 1..45: mol_type = other DNA
gctaatcattgcaagcagtggtatcaacgcagagtacatrgrgrg

SEQ ID NO: 7 - DNA, 23 bp, synthetic construct
  source 1..23: mol_type = other DNA
aagcagtggtatcaacgcagagt

SEQ ID NO: 8 - protein, 16 aa, synthetic construct
MGKIEESKHHHHHHGS